(19) United States
(12) Patent Application Publication
(10) Pub. No.: US 2026/0103721 A1
(43) Pub. Date: Apr. 16, 2026

(54) GENES CONTROLLING BARRIER FORMATION IN ROOTS

(71) Applicant: The Regents of the University of California, Oakland, CA (US)

(72) Inventors: Siobhan BRADY, Davis, CA (US); Concepcion MANZANO FERNANDEZ, Davis, CA (US)

(21) Appl. No.: 19/120,736

(22) PCT Filed: Oct. 12, 2023
(86) PCT No.: PCT/US2023/035047

§ 371 Date: Apr. 11, 2025

Related U.S. Application Data
(60) Provisional application No. 63/415,863, filed on Oct. 13, 2022.

(51) Int. Cl. C12N 15/82; C12N 9/22; C12N 15/11

(52) U.S. Cl. CPC C12N 15/8267; C12N 9/226; C12N 15/11; C12N 15/8202; C12N 15/8205; C12N 15/8213; C12N 2310/20

(57) ABSTRACT

Provided herein are plants having enhanced lignification of root exodermis and methods of generating such plants.

### CROSS-REFERENCE TO RELATED APPLICATION

The present application claims priority to U.S. Provisional Pat. Appl. No. 63/415,863, filed on Oct. 13, 2022, which application is incorporated herein by reference in its entirety.

### BACKGROUND OF THE INVENTION

Plant roots anchor the plant and absorb water and nutrients. As plants are sessile, the control of external mineral ions and water entry is essential for plant survival. In vascular plants, the endodermis is the innermost root cell layer that controls apoplastic movement to and from the plant vasculature by the formation of a barrier that seals the intercellular (apoplastic) space. A first step in endodermal differentiation is the formation of the Casparian strip (CS) which controls the free diffusion of solutes from the soil and prevents the backflow of ions that move into the stele (Enstone, Peterson, and Ma 2002). In Arabidopsis thaliana, specification of endodermis identity occurs within the cortex-endodermis stem cell niche (Helariutta et al. 2000). In addition, the transcription factor SHORT-ROOT (SHR) acts cell non-autonomously to control cell fate across the root radius. SHR protein moves from the stele (vascular cylinder and pericycle) to the adjacent endodermal layer (Nakajima et al. 2001). Nuclear accumulation of SHR is controlled by a feedforward loop whereby it activates transcription of its downstream target, the transcription factor SCARECROW (SCR), within the endodermis (Nakajima et al. 2001; Sabatini et al. 2003; Levesque et al. 2006). SCR then activates its own transcription and physically interacts with SHR, trapping SHR in the nucleus (Cruz-Ramirez et al. 2012; Clark et al. 2019). A subsequent transcriptional cascade determines endodermal cell differentiation (Benfey et al. 1993; Gallagher et al. 2004; Helariutta et al. 2000; Nakajima et al. 2001).

The developmental framework of endodermis differentiation has also been well elucidated in Arabidopsis (Alassimone, Naseer, and Geldner 2010; Roppolo et al. 2011; Hosmani et al. 2013; Y. Lee et al. 2013; Doblas et al. 2017; Nakayama et al. 2017). In the primary differentiation stage, the Casparian strip is formed as a localized impregnation of the primary cell wall with polymerized lignin forming a ring around the central axis of the cell (Naseer et al. 2012). The second differentiation stage is marked by the deposition of suberin lamellae that eventually coat the entire endodermal cell wall surface (Barberon et al. 2016; Geldner 2013). The CASPARIAN STRIP DOMAIN PROTEINS (CASPs) and ENHANCER OF SUBERIN (ESB) proteins are localized to precisely define the Casparian strip domain. They form a platform that is thought to control localized recruitment of proteins involved in CS deposition and function (Alassimone, Naseer, and Geldner 2010; Roppolo et al. 2011; Hosmani et al. 2013; Y. Lee et al, 2013), (Rojas-Murcia et al. n.d.)). The transcription factor MYB36 positively regulates the expression of the CASPI, PER64, and ESB1 genes that are necessary to define Casparian strip positioning as well as its polymerization. Mutation of MYB36 (atmyb36) results in an absent CS (Kamiya et al. 2015; Liberman et al. 2015), ectopic lignin deposition in endodermal cell corners as well as disruption of CS barrier function (Kamiya et al. 2015). The SCHENGEN3/SCHENGEN1/CASPARIAN STRIP INTEGRITY FACTOR2(SGN3/SGN1/CIF2) pathway acts as an elegant surveillance system to perceive defects in CS integrity. If such defects occur, they activate compensatory lignification and suberization (Doblas et al. 2017; Nakayama et al. 2017). Compensatory corner lignification in the armyb36 mutant is mediated by the SGN3/SGN1/CIF pathway as the double mutant atmyb36sgn3 presents a complete loss in endodermis lignification (Reyt et al. 2021). Both the atrmyb36 and the double mutant atmyb36sgn3 have a drastic impact on ion homeostasis and growth (Kamiya et al., 2015; Reyt et al., 2021) proving the important function of the endodermal CS in whole plant growth.

Many plant species also contain an additional cell type that has barrier function, the exodermis or the hypodermis, herein referred to as the exodermis. Located underneath the epidermis, the exodermis is commonly but not invariably present in more than 90% of 200 angiosperms previously examined (Perumalla, Peterson, and Enstone 1990; Peterson and Perumalla 1990; Perumalla, Chmielewski, and Peterson 1990). Using the technology available at this time, it was proposed that the exodermis in these species possess a Casparian strip and/or suberin lamellae. Whether or not the exodermis contains a lignin or suberin-containing barrier in most plant species, is therefore unknown. Most of our knowledge regarding the molecular regulation of endodermal differentiation results from Arabidopsis research. However, Arabidopsis does not develop an exodermis thus our knowledge about its similarities with the endodermis as well as regulation of exodermis differentiation is incomplete.

Cell population translatome profiles and ontology enrichment in domesticated tomato, Solanum lycopersicum, cv. M82 supported a hypothesis that the tomato exodermis is both lignified and suberized which was subsequently experimentally validated (Kajala et al. 2021). The exodermis in tomato has two stages of differentiation: first, the deposition of a polar lignin cap localized on the epidermal face of exodermal cells, followed by suberization surrounding the entire cell surface

### BRIEF SUMMARY OF THE INVENTION

The technical section provided in the present disclosure demonstrated that in tomato, the exodermal barrier is not a Casparian strip, that the polar lignin cap functions as an apoplastic diffusion barrier, and described the developmental timeline by which the this structure forms. It was further demonstrated that while regulation of endodermal Casparian strip differentiation is generally conserved between Arabidopsis and tomato, the regulatory pathway for this first step of exodermal differentiation is genetically distinct. A moderate throughput CRISPR-Cas9 screen identified two transcription factors, SlEXO1 and SISCHIZORIZA (SlSCZ) that collectively restrict deposition of the polar lignin cap to the exodermis; and that SlSCZ additionally regulates the polarity of the lignin cap. Accordingly, mutation of both of these two gene is used for sequestration of carbon in the soil and in breeding plants for drought an salt tolerance.

### BRIEF DESCRIPTION OF THE DRAWINGS

FIG. 1A-H. Exodermal lignin is polarized and serves as an apoplastic barrier. A. Tomato root cross-section stained with fuchsin (pink) and calcofluor (blue) for lignin and cellulose respectively. B. Model for exodermis lignin deposition. C. Transmission electron microscopy (TEM) microscopy of a tomato root section taken from the middle of the root, stained with potassium permanganate (KMnO₄). Left panel=Middle root section. Middle panel: Exodermis. Magnified grey square from left panel. Right panel: Endodermis. Magnified blue square from left panel. ep=epidermis, ex=exodermis, co=cortex, en=endodermis, xy=xylem. D. Same images as in C with an adapted contrast to highlight lignin deposition. E. Tomato root sections from control and treated plants with Piperonylic Acid (PA) for 24 hours. The plants were next incubated in the apoplastic tracer Propidium Iodide (PT) for 30 minutes. F. Quantification of PI blockage at the exodermal lignin cap and penetration into cortex cells in control and PA-treated plants (200 μM) for 24 hours and incubated with PI for 30 minutes (p-value for exodermis PI blockage 9.5e11 and for PI penetration 0.046 n=11. *=statistical significance as determined by ANOVA). G. Tomato root cross-sections from control, PA-treated plants (200 μM) and PA-treated plants with monolignols (200 μM and 20 μM respectively) for 24 hours. The plants were later incubated in the apoplastic tracer PI for 30 minutes. H. Quantification of PI blockage at the epidermis, the exodermal lignin cap and penetration into cortex cells in control and PA-treated plants for 24 hours followed by incubation with PI for 30 minutes (n=11; statistical significance was determined by ANOVA with a post-hoc Tukey HSD test).

FIG. 2A-F. Known endodermal developmental regulators do not control exodermal differentiation. A. Root sections of plants four days old after germination. In wild type, the endodermal layer has a classical Casparian strip composed of polymerized lignin, while the exodermis has a polar lignin cap. Fuchsin (pink) is used to stain lignin, while calcofluor is used to stain the cell walls (blue). The shr-1-9 mutant (right panel) has a missing Casparian Strip earlier in root development time (8 mm from root tip) while ectopic lignin deposition of lignin is observed in cells that surround the vasculature at 10 mm from the root tip. Scale Bar=50 sm. B. Root length of plants four days old after germination. Left panel: root length of wild type and the shr-1-9 mutant (p-value=1.26e06, statistical significance was determined by ANOVA). Middle panel: Symmetry of the cortical layers (including the exodermis), in radial cross-section, was calculated as the minimum number of cortex layers observed divided by the maximum number of cortex layers in wild type and shr-1-9 mutant roots (p-value=9.85e15, statistical significance was determined by ANOVA). Right panel: Symmetry within the vascular cylinder was calculated as the minimum distance from the center of the vascular cylinder to the perimeter of the vascular cylinder, divided by the maximum distance from the center of the vascular cylinder to the perimeter of the vascular cylinder (p-value=2.72e06, statistical significance was determined by ANOVA)(Signif. codes: 0 ‘***’ 0.001 ‘**’ 0.01 ‘*’ 0.05) D. Two independent mutant alleles of SlMYB36 (slmyb36-1 and slmyh36-2) have an endodermis layer with no Casparian strip, but with a wild type polar lignin cap in the exodermis. Left panels=whole root image counterstained with fuchsin; right panels=magnified image of vascular cylinder and endodermis layer. Scale Bar=50 μm D. Mutation of SlSGN3 (slsgn3-1) results in an interrupted non-continuous Casparian strip in the radial axis. Left panels=whole root image counterstained with fuchsin; right panels=magnified image of vascular cylinder and endodermis layer. Small square right panel: Wild-type top view of CS and slsgn3-1 top view of interrupted CS. Asterisks show the discontinuity of the CS in slsgn3-1. Scale Bar=50 μm E. Translational fusions of the SlCASP2 and SlCASP1 proteins under control of their respective promoters, to mCitrine, drive expression specifically in the tomato root endodermis and not in the exodermis. In these longitudinal images, cell walls are stained by calcoflour (blue); lignin is stained by fuchsin (pink) and mCitrine is imaged in the GFP channel (green). Scale Bar=50 μm F. Simultaneous mutation of slcasp1 and slcasp2 showed no defect in endodermal Casparian Strip or exodermal polar lignin cap deposition, relative to wild type. Lignin is stained by fuchsin (pink). Scale Bar=50 μm. All CRISPR-generated mutant or reporter lines were generated by A. tumefaciens transformation unless otherwise noted.

FIG. 3A-H. SlEXO1 and SlSCZ repress lignification in the inner cortical layer(s). A. The evo1-1 mutant allele has an additional lignin polar cap in the first inner cortical layer relative to wild type. The slscz-1 mutant allele has perturbed lignification in the exodermis (either polar lignification, or non-polar lignification) and occasional lignification of the entire cell in the first inner cortical layer. The last exodermis/cortex layer (towards the vasculature) contains a polar lignin cap. Pink=fuchsin/lignin. The slsczslexo1 double mutant has a similar phenotype as the single mutant slsc-1. Scale Bar=50 μm B. SlEXO1 transcriptional fusion. From left to right: Meristem, elongation and maturation zones. SlEXO1 is expressed in the epidermis, exodermis, inner cortex and endodermis in the maturation zone. GFP (Green) and autofluorescence (Blue). Scale Bar=50 μm C. SlEXO1 promoter fused to the SlEXO1 coding region and citrine. D. SlSCZ transcriptional fusion. From left to right: Meristem. elongation and maturation. SlSCZ demonstrates expression in the endodermis, cortex and exodermis meristematic cells and epidermis, exodermis, cortex and endodermis in the maturation zone. Meristem images: Green=GFP. Blue=calcofluor white. Elongation and maturation zone images: Green=GFP. Blue:=autofluorescence. Scale Bar:=50 μm E. SlSCZ promoter fused to the SlSCZ coding region and citrine demonstrates the protein is localized in the stem cell niche, the two inner cortical layers and the exodermis. The SlSCZ protein was not detected at the elongation or maturation zone. Meristem images: Green=citrine. Blue=calcofluor white. Elongation and maturation images: Green=citrine. Blue=autofluorescence. Images are captured from a R. rhizogenes transformed roots. Scale Bar=50 μm E. Root length of wild type and the slexo1 mutant (p-value=0.000465, statistical significance was determined by ANOVA). F Root Length, wild-type, SlEXO1; G, H Root length of wild type and the slscz-1 mutant (p-value=9.55e-07, statistical significance was determined by ANOVA) (Signif. codes: 0 ‘***’ 0.001 ‘**’ 0.01 ‘*’ 0.05).

FIG. 4A-D. SlEX1 and SlSCZ transcriptionally regulate distinct and overlapping genes. A. Principal Component (PC) Analysis of the wild type, slscz/exo2 (green), exo1 (purple) and wild type (orange) R. rhizogenes transformed root transcriptomes. The first two dimensions contribute to 39.5% of the variation. The transcriptomes of these genotypes are distinct in PC 1 and 2 space. B. Venn diagram indicating the common and uniquely differentially expressed genes in two independent exo1 mutant alleles and two independent slscz mutant alleles (FDR=0.05; fold-change=1.6) relative to wild type. C. Heat map indicating significantly differentially expressed genes in each genotype (with slexo1 and slscz representing differentially expressed genes in two independent alleles each) relative to wild type. Color intensity represents the row-normalized z-score. Row clustered with Pearson correlation and columns clustered with Spearman correlation. D. Heat map with the expression of some common genes from Venn diagram from panel B. The selected genes are annotated as peroxidases, receptor kinases, ABC transporters and related with phenylpropanoid metabolism. There is a significant enrichment for peroxidases (Fisher exact test <0.05).

FIG. 5A-B. The exodermal lignin barrier does not compensate for the endodermal Casparian strip. A. Principal component analysis of 24 mineral ions within wild type, myb36-1, exo1-1 and slscz-1 mutant plants (n=4 for slscz-1, slexo1-1 and wild-type; n=3 for slimyb36-1). Considerable variation exists between the ionome of the slscz-1 and slmyb36-1 mutants. B. Log₂ fold change of ions relative to wild type. Heat map indicates the relative abundance of ions. *=statistical significance as determined by ANOVA with a post-hoc Tukey HSD test.

FIG. 6A-C (also referred to as Supplemental FIG. 1 in technical section). The polar lignin cap is conserved in Solanaceae family. A. Cross section of of Capsicum annuum variety Alliance bell pepper stained with fuchsin and calcofluor. B. Cross section of Solanum tuberosum group Andigenum stained with fuchsin and calcofluor. C. Cross section of Nicotiana benthamiana stained with fuschin and calcofluor.

FIG. 7A-B (also referred to as Supplemental FIG. 2 in technical section). The Piperonylic acid concentration tested for lignin biosynthesis inhibition does not affect root growth. A. Schematic representation of the experimental design to test the PA. 4-day-old plants after germination were transferred to MS media+1% sucrose with and without 200 μM of PA. After 24 hours, root growth was measured for the part of the root that grew after transferring. Orange horizontal bar=newly grown root after transferring. B. Root growth after 24H in control and PA treatment for the newly root zone after transferring (statistical significance test determined by ANOVA p-value 0.05).

FIG. 8A-C (also referred to as Supplemental FIG. 3 in technical section). Ultrastructural lignin deposition in the exodermis and endodermis. A. TEM panoramas from wild-type root cross-sections e at 2.5, and 8 mm from the tip, a middle section of the root, and at 1 mm from hypocotyl stained with KMnO₄. Dark deposits indicate electron dense MnO₂ precipitation caused by reaction with lignin. Lower panels show the same pictures with an adapted contrast that highlight the dark deposits. Co=cortex, exo=exodermis, ep=epidermis, en=endodermis, xy=xylem, scale bars=30 μm. B. Close-up of the exodermis area from panels in A (zone defined with brown dashed lines). Lower panels show the same pictures with an adapted contrast that highlight the dark deposits. Dark arrows highlight the lignin deposition in the exodermis cell wall. White arrows highlight the absence of lignin in the exodermis cell wall. Please note that deposition begin at 8 mm from root tip and is oriented toward the epidermis. Co=cortex, exo exodermis, ep=epidermis, scale bars=5 μm. C. Close-up of the endodermis area from panels in A (Zone defined with blue dashed lines). Lower panels show the same pictures with an adapted contrast that highlight the dark deposits. Please note that the Casparian strip is fully lignified already at 5 mm from the root tip. co=cortex, en=endodermis, pe=pericycle, cs=casparian strip, scale bars=5 μm.

FIG. 9A-F (also referred to as Supplemental FIG. 4 in technical section). Endodermal regulators do not regulate exodermal differentiation. A. Hairy root cross sections stained with fuchsin of wild-type (transformed with R. rhizogenes with no binary plasmid) and the mutants shr-1 from 2 independent hairy root lines. Left panel=wild-type, middle panel=shr1-hr1, right panel=shr1-hr2. B. Cross sections of wild-type and the shr-2 mutant allele (stably transformed) from two different parts of the root—8 mm from the tip (middle) and 10 mm from the tip (right). The mutant layer undergoes ectopic lignin deposition in more mature regions of the root. C. Left panel: Radial symmetry of the cortical layers (including the exodermis). This is calculated as the minimum number of cortex layers observed divided by the maximum number of cortex layers in wild type and shr-2 mutant roots (p-value=2.86e06, *=statistical significance as determined by ANOVA n=10-14. D. Symmetry within the vascular cylinder was calculated as the minimum distance from the center of the vascular cylinder to the perimeter of the vascular cylinder, divided by the maximum distance from the center of the vascular cylinder to the perimeter of the vascular cylinder (p-value=1.36e05, *=statistical significance as determined by ANOVA n=10-14). D. Transcriptional fusions for CASP genes; E. SGN3 transcriptional fusion and CIF2 transcriptional fusion. F. Root length in cm of two slmyb36 independent alleles. Statistical significance as determined by ANOVA with a post-hoc Tukey HSD test. n=S G. Mutation of SlSGN3 in the hairy root line slsgn3-hr2f results in an absent Casparian strip, or in a perturbed Casparian strip in the radial axis. Left panels=whole root image counterstained with fuchsin; right panels=magnified image of vascular cylinder and endodermis layer. Scale Bar=50 μm.

FIG. 10A-D (also referred to as Supplemental FIG. 5 in technical section). A previously identified SlMYB36-like gene is not a functional ortholog to AtMYB36. A. MYB36 phylogeny. Phylogenetic trees generated using protein sequences of several plant species. AmTr: Amborella trichopoda, AT: Arabidopsis thaliana, Asparagus: Asparagus officinalis, Azfi: Azolla filiculoides, Bol: Brassica oleracea, Carub: Capsella rubella, CA: Capsicum annuum, Cc: Coffea canephora, Cp: Cucurbita pepo, DCAR: Daucus carota, Gb: Ginkgo biloba, HanXRQ: Helianthus annus, MD: Malus domestica, Mapoly: Marchantia polymorpha, Medtr: Medicago truncatula, Migut: Mimulus guttatus, GSMUA: Musa acuminata, OIT: Nicotiana attenuata, GWHPAAYW: Nymphaea colorata, LOC_Os: Oryra sativa japonica, Peaxi: Petunia axillaris, Pp: Physcomitrella patens, MA: Picea abies, Potri: Populus trichocarpa, Semoe: Selaginella moellendorffii, Seita: Setaria italica, Solyc: Solanum lycopersicum, PGSC: Solanum tuberosum, Sobic: Sorghum bicolor, Thecc: Theobroma cacao, VIT: Vitis vinifera, Zm: Zea mays. B. Detailed MYB36 phylogeny with AMYB36 (At5g57620) highlighted in red, SlMYB36(Solyc07g006750) highlighted in pink, and SlMYB36-b (Solyc04g077260) highlighted in green. C. Expression patterns of two SlMYB36 homologs from the MYB36 phylogeny in individual cell-types from data in (Kajala et al. 2021). Legend on the left represents the Log 2 maximum value across all conditions and legend on the right represent the Log 2(x)/Median. D. Cross section of R. rhizogenes generated mutant allele myb36-b hr8 for Solyc04g077260. Left panel shows whole root cross section and right panel the endodermis magnification from left panel. Scale bar=50 μm.

FIG. 11 (also referred to as Supplemental FIG. 6 in technical section). SlCASP phylogeny. A. Extended SlCASP phylogeny. Phylogenetic trees generated using protein sequences of several plant species. AmTr: Amborella trichopoda, AT: Arabidopsis thaliana, Asparagus: Asparagus officinalis, Azfi: Azolla filiculoides, Bol: Brassica oleracea, Carub: Capsella rubella, CA: Capsicum annuum, Cc: Coffea canephora, Cp: Cucurbita pepo, DCAR: Daucus carota, Gb: Ginkgo biloba, HanXRQ: Helianthus annus, MD: Malus domestica, Mapoly: Marchantia polymorpha, Medtr: Medicago truncatula, Migut: Mimulus guttatus, GSMUA: Musa acuminata, OIT: Nicotiana attenuata, GWHPAAYW: Nymphaea colorata, LOC_Os: Oryza sativa japonica, Peaxi: Petunia axillaris, Pp: Physcomitrella patens, MA: Picea abies, Potri: Populus trichocarpa, Semoe: Selaginella moellendorfii, Seita: Setaria italica, Solyc: Solanum lycopersicum, PGSC: Solanum tuberosum, Sobic: Sorghum bicolor, Thecc: Theobroma cacao, VIT: Vittis vinifera. Zm: Zea mays. B. Detailed phylogeny from Green square in A. AtCASP and SlCASP genes are colored in blue and pink respectively.

FIG. 12 (also referred to as Supplemental FIG. 7 in technical section). SlSGN3 and SlCIF1 phylogeny. A. Extended SlSGN3 phylogeny. Phylogenetic trees generated using protein sequences of several plant species. AmTr: Amborella trichopoda, AT: Arabidopsis thaliana, Asparagus: Asparagus officinalis, Azfi: Azolla filiculoides, Bol: Brassica oleracea, Carub: Capsella rubella, CA: Capsicum annuum, Cc: Coffea canephora, Cp: Cucurbita pepo, DCAR: Daucus carota, Gb: Ginkgo biloba, HanXRQ: Helianthus annus, MD: Malus domestica, Mapoly: Marchantia polymorpha, Medtr: Medicago truncatula, Migut: Mimulus guttatus, GSMUA: Musa acuminata, OT: Nicotiana attenuata, GWHPAAYW: Nymphaea colorata, LOC_Os: Oryza sativa japonica, Peaxi: Petunia axillaris, Pp: Physcomitrella patens, MA: Picea abies, Potri: Populus trichocarpa, Semoe: Selaginella moellendorffii, Seita: Setaria italica, Solyc: Solanum lycopersicum, PGSC: Solanum tuberosum, Sobic: Sorghum bicolor, Thecc: Theobroma cacao, VIT: Vitis vinifera, Zm: Zea mays. B. Detailed phylogeny from green square in A. AtSGN3 and SlSGN3 genes are colored in pink and purple respectively. C. Extended SlCIF1 phylogeny. Phylogenetic trees generated using protein sequences of several plant species. AmTr: Amborella trichopoda, AT: Arabidopsis thaliana, Asparagus: Asparagus officinalis, Azfi: Azolla fidiculoides, Bol: Brassica oleracea, Carub: Capsella rubella, CA: Capsicum annuum, Cc: Coffea canephora, Cp: Cucurbita pepo, DCAR: Daucus carota, Gb: Ginkgo biloba, HanXRQ: Helianthus annus, MD: Malus domestica, Mapoly: Marchantia polymorpha, Medtr: Medicago truncatula, Migut: Mimulus guttatus, GSMUA: Musa acuminata, OIT: Nicotiana attenuata, GWHPAAYW: Nymphaea colorata, LOC_Os: Oryza sativa japonica, Peaxi: Petunia axillaris, Pp: Physcomitrella patens, MA: Picea abies, Potri: Populus trichocarpa, Semoe: Selaginella moellendorffii, Seita: Setaria italica, Solyc: Solanum lycopersicum, PGSC: Solanum tuberosum, Sobic: Sorghum bicolor, Thecc: Theobroma cacao, VIT: Vitis vinifera, Zm: Zea mays. AtCIF1 and AtCIF2 genes are colored in cyan and SlCIF1 is colored in magenta.

FIG. 13 (also referred to as Supplemental FIG. 8 in technical section). The expression of exodermal-enriched transcription factors from Kajala et al., (2021) selected for a hairy root mutant screen. The column names: Epidermis (EP), Exodermis (EXO), Cortex (COR), EN (Endodermis), MCO (Meristematic cortex), Meristematic zone (MZ), Phloem (PH), Vasculature (V), Quiescent center (WOXS), Xylem (XY).

FIG. 14A-B (also referred to as Supplemental FIG. 9 in technical section). SlSCZ phylogeny. A. Extended SlSCZ phylogeny. Phylogenetic trees generated using protein sequences of several plant species. AmTr: Amborella trichopoda, AT: Arabidopsis thaliana, Asparagus: Asparagus officinalis, Azfi: Azolla filiculoides, Bol: Brassica oleracea, Carub: Capsella rubella, CA: Capsicum annuum, Cc: Coffea canephora, Cp: Cucurbita pepo, DCAR: Daucus carota, Gb: Ginkgo biloba, HanXRQ: Helianthus annus, MD: Malus domestica, Mapoly: Marchantia polymorpha, Medtr: Medicago truncatula, Migut: Mimulus guttatus, GSMUA: Musa acuminata, OIT: Nicotiana alternata, GWHPAAYW: Nymphaea colorata, LOC_Os: Oryza saliva japonica, Peaxi: Petunia axillaris, Pp: Physcomitrella patens, MA: Picea abies, Potri: Populus trichocarpa, Semoe: Selaginella moellendorffii, Seita: Setaria italica, Solyc: Solanum lycopersicum, PGSC: Solanum tuberosum, Sobic: Sorghum bicolor, Thecc: Theobroma cacao, VIT: Vitis vinifera, Zm: Zea mays. B. Detailed phylogeny from green square in A. AtSCZ and SlSCZ genes are colored in cyan and pink respectively.

FIG. 15A-U (also referred to as Supplemental FIG. 10 in technical section). CRISPR-edited hairy root mutants for exodermal-enriched transcription factors. Guide RNAs and edited mutations are found in Table 1. For all panels: Root cross sections stained with basic fuchsin. A. Control=Non transformed hairy root. B. slmyc4-1 (Solyc8gf005050). C. slbhlh071-1 (Solyc11g010340). D. slag120-1 (Solyc01g093965). E. slwrk31-1(Solyc05g007110). F. slrngp-1 (Solyc01g099340). G. snyb54-1 (Solyc10g081320). H. slbhlh10-1 (Solyc06g074120). I. slerf3-1(Solyc06g082590). J. slshn1-1 (Solyc01g005630). K. slmyb107b-1 (Solyc02g088190). L. slmyb54-1 (Solyc03g093890). M. CRISPR for mutant Solyc10g080960. N. slmyb107a-1 (Solyc2g079280). O. slathb5-1 (Solyc01g096320). P. slmyb68-1 (Solyc11g069030). Q. slerf98-1 (Solyc05g050790). R. slnuc-1 Solyc09g074780. S. slmyb77-1 (Solyc04g079360). T. slmyb13-1 (Solyc08g008480). U. slnac041 (Solyc01g009860). Scale bar=50 μm.

FIG. 16A N (also referred to as Supplemental FIG. 11 in technical section). slscz and slexo1 hairy root mutant lines. A. Control cross section stained with fuschin. B. slscz hr3 cross section stained with fuchsin. C. slscz hr4 cross section stained with fuchsin. D. slscz hr5 cross section stained with fuchsin. E. slscz hr12 cross section stained with fuchsin. F. slscz hr20 cross section stained with fuchsin. G. Stable transformed slscz-2 line cross section stained with fuchsin. H. Control cross section stained with fuschin. I. slexo1 hr3 cross section stained with fuchsin. J. slexo1 hr6 cross section stained with fuchsin. K. slexo1 hr7 cross section stained with fuchsin. L. slexo1 hr10 cross section stained with fuchsin. M. slexo1 hr19 cross section stained with fuchsin. N. Control cross section stained with fuschin. O. slexoslscz hr11 double mutant cross section stained with fuchsin. Scale bar:=50 μm.

FIG. 17A-E (also referred to as Supplemental FIG. 12 in technical section). DEG in slexo1 and slscz hairy root mutants and GO categories. A. Volcano plot with the DEG from slexo1 and slscz hairy root mutants. B. Enriched GO terms in slexo1 for upregulated genes relative to wild type and downregulated genes relative to wild type (FC 1.3 p-value <0.05). C. Enriched GO terms in slscz for upregulated genes relative to wild type and downregulated genes relative to wild type (FC 1.3 p-value <0.05). D. UpSet diagram indicating overlap between each of the genotypes for upregulated genes relative to wild type and downregulated genes relative to wild type, with the same FDR and fold-change in C. E. GO categories from the common upregulated genes relative to wild type and downregulated genes relative to wild type (FC 1.3 p-value <0.05) from slexo1 and slscz presented in the Venn diagram from panel B.

FIG. 18A-B (also referred to as Supplemental FIG. 13 in technical section). DEG from slexo1 and slscz hairy root mutants are enriched in epidermis, exodermis and cortex. A. Enrichment of the DEG in slexo1 hairy root mutants in cell-type data from Kajala et al. 2021 dataset. B. Enrichment of the DEG in slscz hairy root mutants in cell-type data from Kajala et al. 2021 dataset. The cell types abbreviation names: General cortex (gCOR), Inner cortex (i-COR), MCO (Meristematic cortex), Meristematic zone (MZ), Phloem (PH), Vasculature (V), Quiescent center (QC).

FIG. 19 (also referred to as Supplemental FIG. 14 in technical section). Ion content in slexo1-1, slscz-1, slmyb36-1 and wild-type. A. Ion content in ppm (Parts per million) for As (Arsenic), B (Boron), Ca (Calcium), Cd (Cadmium), Co (Cobalt), Cu (Cupper), Fe (Iron), K (Potassium), Li (Lithium), Mg (Magnesium), Mn (Manganese), Mo (Molibdenum), Na (Sodium), Ni (Nikel), P (Phosphorous), Rb (Rubidium), S (Sulfur), Se (Selenium), Sr (Strontium), and Zn (Zinc). Statistical significance was determined by ANOVA with a post-hoc Tukey HSD test.

### DETAILED DESCRIPTION OF THE INVENTION

The present disclosure provides plants having enhanced exodermal lignin deposition. In some embodiments, such plants have enhanced drought or salt tolerance. Such plants are genetically modified to decrease levels of a C2H2 transcription factor-9 (also referred to herein as an SlEXO gene, which designation is derived from reference to a tomato, but also refers a homolog of an SlEXO gene from a non-tomato plant)) and a heat shock transcription factor B4 gene (SCHIZORIZA, also referred to herein as an Scz gene).

The term “enhanced drought tolerance” or “increased drought tolerance” of a plant refers to any measurable improvement in a physiological or physical characteristic, such as yield, as measured relative to a reference or control plant when grown under drought conditions. In some embodiments, the reference or control plant is a wildtype plant. In some embodiments, the reference or control plant is a plant of the same genetic background that does not have the genetic modification. In some embodiments, a plant genetically modified as described herein has increased tolerance to drought and increased tolerance to salt.

The term “enhanced salt tolerance” or “increased salt tolerance” of a plant refers to any measurable improvement in a physiological or physical characteristic, such as yield, as measured relative to a reference or control plant when grown under high salt conditions. In some embodiments, the reference or control plant is a wildtype plant. In some embodiments, the reference or control plant is a plant of the same genetic background that does not have the genetic modification.

The term “SlEXO transcription factor” or “SlEXO polypeptide” refers to a polypeptide encoded by a tomato C2H2 transcription factor gene available under accession number Solyc09g011120 (see, also NCBI GENE ID: 101261848 zinc finger protein 10 (Solanum lycopersicum)), or a homolog, or ortholog, thereof in another plant. An illustrative C2H2 polypeptide is provided in SEQ ID NO:1. An illustrative cDNA sequence encoding the polypeptide is provided in SEQ ID NO:2. In some embodiments, an endogenous gene encoding a SlEXO having at least 50%, 55%, 60%, 65%, 70%, 75%, 80%, 85%, 90% or 95% to SEQ ID NO:1 is targeted for inhibition. In some embodiments a SlEXO gene of a Solanaceous plant is targeted for inhibition. Illustrative SlEXO homologs are provided in Table 3.

The term “SCZ transcription factor” or “SCZ polypeptide” refers to a polypeptide encoded by a plant SCHIZORIZA heat shock transcription factor gene, or homolog, or ortholog, thereof in another plant. An illustrative polypeptide sequence is available under accession number Solyc04g078770 (synonym SGN-U603561). See, also NCBI Accession number XM_004238096.4. Arabidopsis homolog nucleic acid and polypeptide sequences can be found through accession number At1g46264 (see, e.g., polypeptide sequence available under AT1AG46264.1 and listing of ortholog sequences from alternative plants). An illustrative tomato SCZ polypeptide is provided in SEQ ID NO:3. An illustrative cDNA sequence encoding the polypeptide is provided in SEQ ID NO:4. In some embodiments, an endogenous gene encoding a C2H2 having at least 50%, 55%, 60%, 65%, 70%, 75%, 80%, 85%, 90% or 95% to SEQ ID NO:3 is targeted for inhibition. In some embodiments an SCZ gene of a Solanaceous plant is targeted for inhibition. Illustrative SCZ homologs are provided in Table 2.

The invention employs various routine recombinant nucleic acid techniques. Generally, the nomenclature and the laboratory procedures in recombinant DNA technology described below are commonly employed in the art. Many manuals that provide direction for performing recombinant DNA manipulations are available, e.g., Sambrook & Russell, Molecular Cloning, A Laboratory Manual (3rd Ed, 2001); and Current Protocols in Molecular Biology (Ausubel, et al., John Wiley and Sons, New York, 2009-2022).

The term “nucleic acid” or “polynucleotide” refers to a single or double-stranded polymer of deoxyribonucleotide or ribonucleotide bases read from the 5′ to the 3′ end. Nucleic acids may also include modified nucleotides that permit correct read through by a polymerase and do not significantly alter expression of a polypeptide encoded by that nucleic acid.

The phrase “nucleic acid encoding” or “polynucleotide encoding” refers to a nucleic acid which directs the expression of a specific protein or peptide. The nucleic acid sequences include both the DNA strand sequence that is transcribed into RNA and the RNA sequence that is translated into protein. The nucleic acid sequences include both the full length nucleic acid sequences as well as non-full length sequences derived from the full length sequences. It should be further understood that the sequence includes the degenerate codons of the native sequence or sequences which may be introduced to provide codon preference in a specific host cell.

Two nucleic acid sequences or polypeptides are said to be “identical” if the sequence of nucleotides or amino acid residues, respectively, in the two sequences is the same when aligned for maximum correspondence as described below. “Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. When percentage of sequence identity is used in reference to proteins or peptides, it is recognized that residue positions that are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Bio. Sci. 4:11-17 (1988) e.g., as implemented in the program PC/GENE (Intelligenetics, Mountain View, California, USA).

The term “substantial identity” or “substantially identical,” as used in the context of polynucleotide or polypeptide sequences, refers to a sequence that has at least 50% sequence identity to a reference sequence. Alternatively, percent identity can be any integer from 50% to 100%. Exemplary embodiments include at least: 50%, 55%, 60%, 65%, 70%, 75%, 80%, 85%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith and Waterman Add. APL. Mah. 2:482 (1981), by the homology alignment algorithm of Needleman and Wunsch J. Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)). For purposes of this application, amino acid sequence identity is determined using BLASTP with default parameters.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The term “complementary to” is used herein to mean that a polynucleotide sequence is complementary to all or a portion of a reference polynucleotide sequence. In some embodiments, a polynucleotide sequence is complementary to at least 15, at least 20, at least 25, at least 30, at least 40, at least 50, at least 75, at least 100, at least 125, at least 150, at least 175, at least 200, or more contiguous nucleotides of a reference polynucleotide sequence. In some embodiments, a polynucleotide sequence is “substantially complementary” to a reference polynucleotide sequence if at least 70%, at least 75%, at least 80%, at least 85%, at least 90%, or at least 95% of the polynucleotide sequence is complementary to the reference polynucleotide sequence.

A polynucleotide sequence is “heterologous” to an organism or a second polynucleotide sequence if it originates from a foreign species, or, if from the same species, is modified from its original form. For example, when a promoter is said to be operably linked to a heterologous coding sequence, it means that the coding sequence is derived from one species whereas the promoter sequence is derived another, different species; or, if both are derived from the same species, the coding sequence is not naturally associated with the promoter (e.g., is a genetically engineered coding sequence, e.g., from a different gene in the same species, or an allele from a different ecotype or variety).

An “expression cassette” refers to a nucleic acid construct that, when introduced into a host cell, results in transcription and/or translation of an RNA or polypeptide, respectively. Antisense or sense constructs that are not or cannot be translated are expressly included by this definition. In the case of both expression of transgenes and suppression of endogenous genes (e.g., by antisense, or sense suppression) one of skill will recognize that the inserted polynucleotide sequence need not be identical, but may be only substantially identical to a sequence of the gene from which it was derived.

The term “promoter,” as used herein, refers to a polynucleotide sequence capable of driving transcription of a coding sequence in a cell. Thus, promoters used in the polynucleotide constructs of the invention include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. These cis-acting sequences typically interact with proteins or other biomolecules to carry out (turn on/off, regulate, modulate, etc.) gene transcription. A “plant promoter” is a promoter capable of initiating transcription in plant cells. A “constitutive promoter” is one that is capable of initiating transcription in nearly all tissue types, whereas a “tissue-specific promoter” initiates transcription only in one or a few particular tissue types. An “inducible promoter” is one that initiates transcription only under particular environmental conditions or developmental conditions.

The term “plant” includes whole plants, shoot vegetative organs and/or structures (e.g., leaves, stems and tubers), roots, flowers and floral organs (e.g., bracts, sepals, petals, stamens, carpels, anthers), ovules (including egg and central cells), seed (including zygote, embryo, endosperm, and seed coat), fruit (e.g., the mature ovary), seedlings, plant tissue (e.g., vascular tissue, ground tissue, and the like), cells (e.g., guard cells, egg cells, trichomes and the like), and progeny of same.

### Inhibition of SlEXO1 and SCZ

Expression or activity of an SlEXO1 target gene and SCZ target gene in a plant can be inhibited, knocked out, mutated or engineered to decrease expression. For example, in some embodiments, the native gene sequence mutated, knocked out, or engineered in a plant encodes a polypeptide identical or substantially identical (e.g., at least 50, 55, 60, 65, 70, 75, 80, 85, 90, or 95% identical) to protein sequence of SEQ ID NO:1 or SEQ ID NO:3). Gene sequences can be readily identified in many plant species in view of known genome sequences and the conserved nature of the proteins. A plant engineered to inhibit expression or activity of an SlEXO1 target gene and SCZ target gene as described herein typically has enhanced drought tolerance and/or enhanced salt tolerance compared to a reference control plant, e.g., wildtype plant or plant of the same genetic background without the genetic modifications to inhibition expression or activity of the two genes, when tested under drought and/or high-salt conditions. In some embodiments, e.g., a solanaceous plant such as tomato, the plant exhibits increased lignification of the root exodermis compared to a reference control plant.

In some embodiments, the gene sequence is knocked out in the plant. “Knocked out” means that the plant does not make the particular protein encoded by the gene. Knockouts can be achieved in a variety of ways. For the purposes of this document, a knock out can be achieved by a deletion of all or a substantial part (e.g., majority) or the coding sequence for a polypeptide identical or substantially identical to an SlEXO1 and SCZ polypeptide sequence. Alternatively a knock out can be achieved by introduction of a mutation that prevents translation or transcription (e.g., a mutation that introduces a stop codon early in the coding sequence or that disrupts transcription). A knock out can also be achieved by silencing or other suppression methods, e.g., such that the plant expresses substantially less of the native protein (e.g., less than 50, 25, 10, 5, or 1% of native expression).

In some embodiments, the mutation introduced into the protein is a single amino acid change that reduces or eliminates the protein's activity. Alternatively, the mutation can include any number (e.g., 1, 2, 3, 4, 5, 6, 7, 8, 9, or 10 or more) of amino acid changes, deletions or insertions that reduce or eliminate the protein activity.

Methods for introducing genetic mutations into plant genes and selecting plants with desired traits are well known and can be used to introduce mutations or to knock out a protein. For instance, seeds or other plant material can be treated with a mutagenic insertional polynucleotide (e.g., transposon, T-DNA, etc.) or chemical substance, according to standard techniques. Such chemical substances include, but are not limited to, the following: diethyl sulfate, ethylene imine, ethyl methanesulfonate and N-nitroso-N-ethylurea. Alternatively, ionizing radiation from sources such as, X-rays or gamma rays can be used. Plants having mutated protein can then be identified, for example, by phenotype or by molecular techniques.

Modified protein chains can also be readily designed utilizing various recombinant DNA techniques well known to those skilled in the art and described for instance, in Sambrook et al., supra. Hydroxylamine can also be used to introduce single base mutations into the coding region of the gene (Sikorski et al., Meth. Enzymol., 194:302-318 (1991)). For example, the chains can vary from the naturally occurring sequence at the primary structure level by amino acid substitutions, additions, deletions, and the like. These modifications can be used in a number of combinations to produce the final modified protein chain.

Alternatively, homologous recombination can be used to induce targeted gene modifications or knockouts by specifically targeting the gene in vivo (see, generally, Grewal and Klar, Genetics, 146:1221-1238 (1997) and Xu et al., Genes Dev., 10:2411-2422(1996)). Homologous recombination has been demonstrated in plants (Puchta et al., Experientia, 50:277-284 (1994); Swoboda et al., EMBO J., 13:484-489 (1994); Offringa et al., Proc. Natl. Acad. Sci. USA, 90:7346-7350 (1993); and Kempin et al., Nature, 389:802-803 (1997)).

In applying homologous recombination technology to a gene, mutations in selected portions of gene sequences (including 5′ upstream, 3′ downstream, and intragenic regions) can be made in vitro and then introduced into the desired plant using standard techniques. Since the efficiency of homologous recombination is known to be dependent on the vectors used, use of dicistronic gene targeting vectors as described by Mountford et al., Proc. Natl. Acad. Sci. USA, 91:4303-4307 (1994); and Vaulont et al., Transgenic Res., 4:247-255 (1995) are conveniently used to increase the efficiency of selecting for altered protein gene expression in transgenic plants. The mutated gene will interact with the target wild-type gene in such a way that homologous recombination and targeted replacement of the wild-type gene will occur in transgenic plant cells, resulting in suppression of target protein activity.

Any of a number of genome editing proteins known to those of skill in the art can be used to mutate or knock out the target protein. The particular genome editing protein used is not critical, so long as it provides site-specific mutation of a desired nucleic acid sequence. Exemplary genome editing proteins include targeted nucleases such as engineered zinc finger nucleases (ZFNs), transcription-activator like effector nucleases (TALENs), and engineered meganucleases. In addition, systems which rely on an engineered guide RNA (a gRNA) to guide an endonuclease to a target cleavage site can be used. The most commonly used of these systems is the CRISPR/Cas system with an engineered guide RNA to guide the Cas-9 endonuclease to the target cleavage site.

CRISPR (Clustered Regularly Interspaced Short Palindromic Repeats)/Cas (CRISPR-associated) system, are adaptive defense systems in prokaryotic organisms that cleave foreign DNA. CRISPR loci in microbial hosts contain a combination of CRISPR-associated (Cas) genes as well as non-coding RNA elements which determine the specificity of the CRISPR-mediated nucleic acid cleavage. Three types (1-III) of CRISPR systems have been identified across a wide range of bacterial hosts. In the typical system, a Cas endonuclease (e.g., Cas9) is guided to a desired site in the genome using small RNAs that target sequence-specific single- or double-stranded DNA sequences. The CRISPR/Cas system has been used to induce site-specific mutations in plants (see Miao et al. 2013 Cell Research 23:1233-1236).

The basic CRISPR system uses two non-coding guide RNAs (crRNA and tracrRNA) which form a crRNA:tracrRNA complex that directs the nuclease to the target DNA via Wastson-Crick base-pairing between the crRNA and the target DNA. Thus, the guide RNAs can be modified to recognize any desired target DNA sequence. In some embodiments, a Cas nuclease is targeted to the target gene location with a chimeric single-guide RNA (sgRNA) that contains both the crRNA and tracRNA elements.

Zinc finger nucleases (ZFNs) are engineered proteins comprising a zinc finger DNA-binding domain fused to a nucleic acid cleavage domain, e.g., a nuclease. The zinc finger binding domains provide specificity and can be engineered to specifically recognize any desired target DNA sequence. For a review of the construction and use of ZFNs in plants and other organisms, see Urnov et al. 2010 Nat Rev Genet. 11(9):636-46.

Transcription activator like effectors (TALEs) are proteins secreted by certain species of Xanthomonas to modulate gene expression in host plants and to facilitate bacterial colonization and survival. TALEs act as transcription factors and modulate expression of resistance genes in the plants. Recent studies of TALEs have revealed the code linking the repetitive region of TALEs with their target DNA-binding sites. TALEs comprise a highly conserved and repetitive region consisting of tandem repeats of mostly 33 or 34 amino acid segments. The repeat monomers differ from each other mainly at amino acid positions 12 and 13. A strong correlation between unique pairs of amino acids at positions 12 and 13 and the corresponding nucleotide in the TALE-binding site have been found. The simple relationship between amino acid sequence and DNA recognition of the TALE binding domain allows for the design DNA binding domains of any desired specificity.

TALEs can be linked to a non-specific DNA cleavage domain to prepare genome editing proteins, referred to as TALENs. As in the case of ZFNs, a restriction endonuclease, such as FokI. can be conveniently used. For a description of the use of TALENs in plants, see Mahfouz et al. 2011 Proc Natl Acad Sci USA. 108:2623-8 and Mahfouz 2011 GM Crops. 2:99-103.

Meganucleases are endonucleases that have a recognition site of 12 to 40 base pairs. As a result, the recognition site occurs rarely in any given genome. By modifying the recognition sequence through protein engineering, the targeted sequence can be changed and the nuclease can be used to cleave a desired target sequence. (See Seligman, er al. 2002 Nucleic Acids Research 30: 3870-9 WO06097853, WO06097784, WO04067736, or US20070117128).

In addition to the methods described above, other methods for introducing genetic mutations into plant genes and selecting plants with desired traits are known. For instance, seeds or other plant material can be treated with a mutagenic chemical substance, according to standard techniques. Such chemical substances include, diethyl sulfate, ethylene imine, ethyl methanesulfonate (EMS) and N-nitroso-N-ethylurea. Alternatively, ionizing radiation from sources such as, X-rays or gamma rays can be used.

Also provided are methods of suppressing expression or activity of a SlEXO1 and SCZ polypeptide in a plant, e.g., a polypeptide substantially identical to a protein of SEQ ID NO:1 or SEQ ID NO:3 in a plant using expression cassettes that comprise RNA molecules (or fragments thereof) that inhibit endogenous target expression or activity in a plant cell. Suppressing or silencing gene function refers generally to the suppression of levels of mRNA or protein expressed by the endogenous gene and/or the level of the protein functionality in a cell. The terms do not require specific mechanism and could include RNAi (e.g., short interfering RNA (siRNA) and microRNA (miRNA)), anti-sense, cosuppression, viral-suppression, hairpin suppression, stem-loop suppression, and the like.

A number of methods can be used to suppress or silence gene expression in a plant. The ability to suppress gene function in a variety of organisms, including plants, using double stranded RNA is well known. Expression cassettes encoding RNAi typically comprise a polynucleotide sequence at least substantially identical to the target gene linked to a complementary polynucleotide sequence. The sequence and its complement are often connected through a linker sequence that allows the transcribed RNA molecule to fold over such that the two sequences hybridize to each other.

RNAi (e.g., siRNA, miRNA) appears to function by base-pairing to complementary RNA or DNA target sequences. When bound to RNA, the inhibitory RNA molecules trigger either RNA cleavage or translational inhibition of the target sequence. When bound to DNA target sequences, it is thought that inhibitory RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides in length that are processed from longer precursor transcripts that form stable hairpin structures.

In addition, antisense technology can be conveniently used. To accomplish this, a nucleic acid segment at least substantially identical to the desired gene is cloned and operably linked to a promoter such that the antisense strand of RNA will be transcribed. The expression cassette is then transformed into a plant and the antisense strand of RNA is produced. In plant cells, it has been suggested that antisense RNA inhibits gene expression by preventing the accumulation of mRNA which encodes the protein of interest.

Another method of suppression is sense suppression. Introduction of expression cassettes in which a nucleic acid is conFig.d in the sense orientation with respect to the promoter has been shown to be an effective means by which to block the transcription of target genes.

For these techniques, the introduced sequence in the expression cassette need not have absolute identity to the target gene. In addition, the sequence need not be full length, relative to either the primary transcription product or fully processed mRNA. One of skill in the art will also recognize that using these technologies families of genes can be suppressed with a transcript. For instance, if a transcript is designed to have a sequence that is conserved among a family of genes, then multiple members of a gene family can be suppressed. Conversely, if the goal is to only suppress one member of a homologous gene family, then the transcript should be targeted to sequences with the most variance between family members.

Gene expression can also be inactivated using recombinant DNA techniques by transforming plant cells with constructs comprising transposons or T-DNA sequences. Mutants prepared by these methods are identified according to standard techniques. For instance, mutants can be detected by PCR or by detecting the presence or absence mRNA, e.g., by northern blots or reverse transcription PCR (RT-PCR).

Catalytic RNA molecules or ribozymes can also be used to inhibit expression of embryo-specific genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA cleaving activity upon them, thereby increasing the activity of the constructs. The design and use of target RNA-specific ribozymes is well known.

The recombinant construct encoding a genome editing protein or a nucleic acid that suppresses expression may be introduced into the plant cell using standard genetic engineering techniques, well known to those of skill in the art. In the typical embodiment, recombinant expression cassettes can be prepared according to well-known techniques. In the case of CRISPR/Cas nuclease, the expression cassette may transcribe the guide RNA, as well.

In some embodiments, the genome editing protein itself, is introduced into the plant cell. In these embodiments, the introduced genome editing protein is provided in sufficient quantity to modify the cell but does not persist after a contemplated period of time has passed or after one or more cell divisions. In such embodiments, no further steps are needed to remove or segregate away the genome editing protein and the modified cell.

in these embodiments, the genome editing protein is prepared in vitro prior to introduction to a plant cell using well known recombinant expression systems (bacterial expression, in vitro translation, yeast cells, insect cells and the like). After expression, the protein is isolated, refolded if needed, purified and optionally treated to remove any purification tags, such as a His-tag. Once crude, partially purified, or more completely purified genome editing proteins are obtained, they may be introduced to a plant cell via electroporation, by bombardment with protein coated particles, by chemical transfection or by some other means of transport across a cell membrane.

Plant expression cassettes for expression of siRNA or gene editing proteins as described herein can contain the polynucleotide operably linked to a promoter (e.g., one conferring inducible or constitutive, environmentally- or developmentally-regulated, or cell- or tissue-specific/selective expression), a transcription initiation start site, a ribosome binding site, an RNA processing signal, a transcription termination site, and/or a polyadenylation signal.

A number of promoters can be used. A plant promoter fragment can be employed which will direct expression of the desired polynucleotide in all tissues of a plant. In some embodiments, promoters described herein comprise from 100-bp to 2 kb region upstream (5′) from where gene transcription is initiated.

In some embodiments, the promoter is a “constitutive” promoter active under most environmental conditions and state of development or cell differentiation. Examples of constitutive promoters include the cauliflower mosaic virus (CaMV) 35S transcription initiation region.

Alternatively, the plant promoter can direct expression of the polynucleotide under environmental control. Such promoters are referred to here as “inducible” promoters. Environmental conditions that may affect transcription by inducible promoters include biotic stress, abiotic stress, saline stress, drought stress, pathogen attack, anaerobic conditions, cold stress, heat stress, hypoxia stress, or the presence of light.

In addition, chemically inducible promoters can be used. Examples include those that are induced by benzyl sulfonamide, tetracycline, abscisic acid, dexamethasone, ethanol or cyclohexenol.

Examples of promoters under developmental control include promoters that initiate transcription only, or preferentially, in certain tissues such as roots or specific plant tissue such as exodermis.

In some embodiments, the promoter directs expression in the exodermis, which are internal root tissues of the plant.

Illustrative promoters in solanaceous plants such as tomato, include, but are not limited to exodermis-enriched promoters such as one of the following:
Solyc12g005785, Solyc08g066890, Solyc07g049460, Solyc08g081555, Solyc09g082530, Solyc04g081860, Solyc07g052530, Solyc00g095860, Solyc08g078920, Solyc07g052540, Solyc0g076240, Solyc01g111230, Solyc08g075830, Solyc09g065430, Solyc12g006110, Solyc00g072400, Solyc10g076243, Solyc09g074890, Solyc07g047740, Solyc06g064960, Solyc05g012580, Solyc10g037880, Solyc12g096620, Solyc11g072110, Solyc08g066930, Solyc01g081250, Solyc03gW05760, Solyc02g084850, Solyc10g007930, Solyc05g046020, Solyc12g049680, Solyc02g070080, Solyc06g060620, Solyc08g081780, Solyc12g011030, Solyc09g075670, Solyc11g012360, Solyc07g049240, Solyc10g009150, Solyc03g096420, Solyc08g074682, Solyc05g007470, Solyc12g097080, Solyc06g011350, Solyc08g014000, Solyc08g068780, Solyc09g082270, Solyc06g067870, Solyc08g061970, Solyc11g066270, Solyc08g079190, Solyc07g055060, Solyc02g092670, Solyc03g115690, Solyc09g007770, Solyc10g085880, Solyc03g120475, Solyc02g065780, Solyc08g066880, Solyc01g090610, Solyc01g066910, Solyc01g108860, Solyc10g083460, Solyc11g031950, Solyc08g008050, Solyc04g007400, Solyc11g011190, Solyc02g080200, Solyc06g060760, Solyc04g077670, Solyc08g079200, Solyc06g066830, Solyc09g089830, Solyc04g007750, Solyc12g009650, Solyc09g072590, Solyc03g096030. Solyc06g073460, Solyc07g043130, Solyc02g089250, Solyc09g098620, Solyc09g007760, Solyc01g109500, Solyc11g013810, Solyc06g060070, Solyc08g005960, Solyc06g075360, Solyc08g081190, Solyc01g096420, Solyc06g075650, Solyc12g005940, Solyc09g008320, Solyc12g056800, Solyc12g013690, Solyc02g086880, Solyc01g105410, Solyc09g014280, Solyc12g087940, Solyc03g111310. Solyc01g106780, Solyc01g097520, Solyc07g016215, Solyc02g080640, or Solyc02g081400. Promoter designations are from Sol Genomics Network database, genome version Sl 3.0.

In some embodiments, the promoter may be a drought-inducible promoter, e.g., from a solanaceous plant, e.g., such as tomato. In some embodiments, the solanaceous promoter may be one of the following:
Solyc06g076760, Solyc03g025810, Solyc12g010545, Solyc03g007230, Solyc12g006050, Solyc12g008430, Solyc02g086530, Solyc09g015070, Solyc12g089350, Solyc06g048860, Solyc06g068160, Solyc01g096320, Solyc11g071350, Solyc09g090790, Solyc09g082290, Solyc01g100090, Solyc02g090210, Solyc05g053160, Solyc01g060260, Solyc10g008700, Solyc01g006620, Solyc04g011600, Solyc03g006360, Solyc03g117800, Solyc11g067190. Solyc01g109920, Solyc09g097760, Solyc06g060970, Solyc08g067260, Solyc05g010330, Solyc03g112590, Solyc06g067980, Solyc0g078770, Solyc01g057000, Solyc8g078550, Solyc01g111040, Solyc12g009680, Solyc03g097585, Solyc01g087180, Solyc09g082550, Solyc01g103060, Solyc02g079640, Solyc07g055560, Solyc02g072540, Solyc11g009100, Solyc11g066700, Solyc08g079270, Solyc12g098900, Solyc06g076800, Solyc09g082340, Solyc06g060970, Solyc09g082280, Solyc03g097620, Solyc03g019820, Solyc01g099880, Solyc01g095320, Solyc09g015070, Solyc03g025810, Solyc06g051860, Solyc03g006360, Solyc11g007807, Solyc12g006050, Solyc12g098900, Solyc02g084850, Solyc02g061800, Solyc09g090800, Solyc10g079150, Solyc01g109920, Solyc03g044600, Solyc03g065250, Solyc08g081740, Solyc10g083690, Solyc03g097600, Solyc06g069070, Solyc04g071770, Solyc01g095305, Solyc01g096320, Solyc08g062960, Solyc03g095650, Solyc09g082300, Solyc03g007790, Solyc03g096670, Solyc08g078757, Solyc03g007230, Solyc03g013440, Solyc06g050800, Solyc08g075150, Solyc10g008700, Solyc04g016430, Solyc04g007470, Solyc10g024490, Solyc06g076400, Solyc09g083050, Solyc01g109810, Solyc01g057000, Solyc06g008580, Solyc08g068150, Solyc09g005610, Solyc12g010545, or Solyc04g072700.

Also provided are methods of suppressing expression or activity of a polypeptide substantially identical to SEQ ID NO:1 or SEQ ID NO:3 in a plant using an upstream open reading frame (uORF) to reduce expression at the level of mRNA translation.

Methods for transformation of plant cells are well known in the art, and the selection of the most appropriate transformation technique for a particular embodiment of the invention may be determined by the practitioner. Suitable methods may include electroporation of plant protoplasts, liposome-mediated transformation, polyethylene glycol (PEG) mediated transformation, transformation using viruses, micro-injection of plant cells, micro-projectile bombardment of plant cells, and Agrobacterium tumefaciens or Rhizobium rhizogenes-mediated transformation. Transformation means introducing a nucleotide sequence in a plant in a manner to cause stable or transient expression of the sequence.

In some embodiments, in planta transformation techniques (e.g., vacuum-infiltration, floral spraying or floral dip procedures) are used to introduce the expression cassettes of the invention (typically in an Agrobacterium vector) into meristematic or germline cells of a whole plant. Such methods provide a simple and reliable method of obtaining transformants at high efficiency while avoiding the use of tissue culture. (see, e.g., Bechtold et al. 1993 C. R. Acad. Sci. 316:1194-1199; Chung et al. 2000 Transgenic Res. 9:471-476; Clough et al. 1998 Plant J. 16:735-743; and Desfeux et al. 2000 Plant Physiol 123:895-904). In these embodiments, seed produced by the plant comprise the expression cassettes encoding the proteins. The seed can be selected based on the ability to germinate under conditions that inhibit germination of the untransformed seed.

If transformation techniques require use of tissue culture, transformed cells may be regenerated into plants in accordance with techniques well known to those of skill in the art. The regenerated plants may then be grown. and crossed with the same or different plant varieties using traditional breeding techniques to produce seed, which are then selected under the appropriate conditions.

An expression cassette can be integrated into the genome of the plant cells, in which case subsequent generations will express the encoded proteins. Alternatively, the expression cassette is not integrated into the genome of the plants cell, in which case the encoded protein is transiently expressed in the transformed cells and is not expressed in subsequent generations.

Any plant can be modified as described herein to have modulated amounts of suberin. Exemplary plants include species from the genera Arachis, Asparagus, Atropa, Atven, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Mahis, Manihot, Majorana, Medicago, Nicotiana, Oryza, Panieum, Pennisetum, Persea, Pisum, Pyrus, Prunus, Raphanus, Secale, Senecio, Sinapis, Solanum, Sorghum, Trigonella, Triticum, Vitis, Vigna, and Zea. In some embodiments, the plant is a gramineous or solanaceous plant. Exemplary gramineous plants include but are not limited to rice, maize, wheat, sorghum, barley, rye, oat, millet, fescue, bluegrass, and ryegrass.

### Techniques

Exodermal Lignification Comprises a Polarly Localized Lignin Cap that Acts as an Apoplastic Barrier

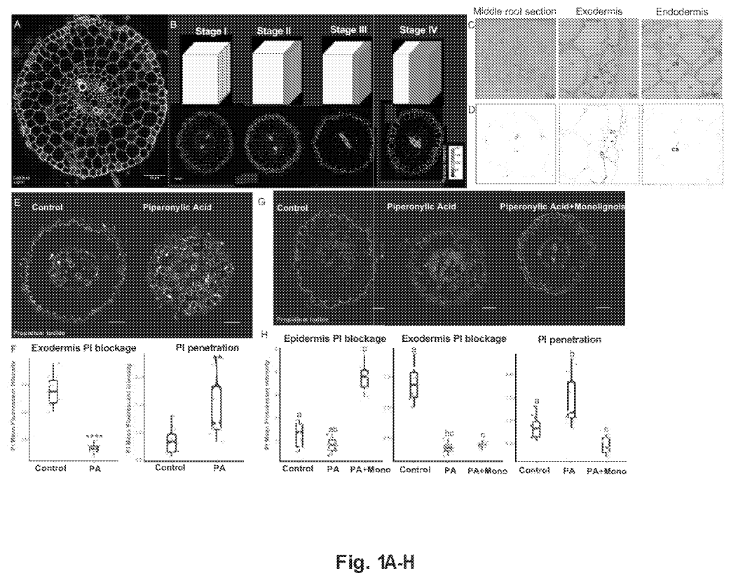

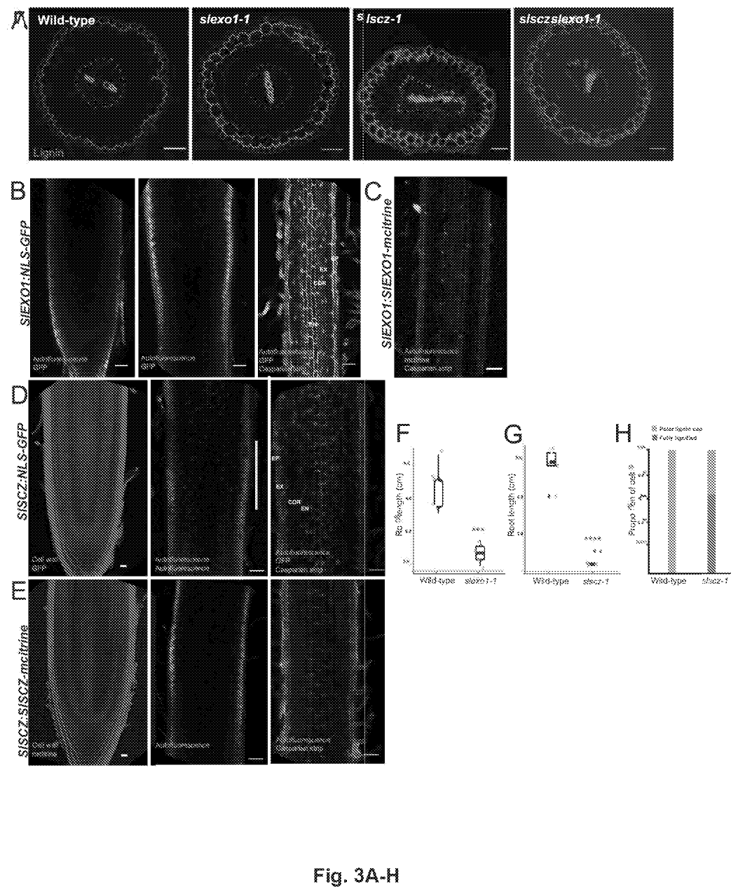

The Casparian strip in the Arabidopsis endodermis is composed of polymerized lignin impregnated in the cell wall, forming a belt surrounding the longitudinal, central axis of the cell. Endodermal lignification occurs in the early root maturation zone where it functions as an apoplastic diffusion barrier (Naseer et al. 2012). Prior research described the exodermis in more than 200 species as possessing a Casparian strip (Peterson and Perumalla 1990; Perumalla, Peterson, and Enstone 1990, Perumalla, Chmielewski, and Peterson 1990). Instead, the tomato exodermis forms a polarized lignin cap on the epidermal facing exodermal cell surface (FIG. 1A). The temporal nature of exodermal lignin deposition was characterized in a five-day old seedling using histochemical staining with basic fuchsin (lignin) and calcoflour white (cellulose) to counterstain the cell walls (FIG. 1A). These data were complemented with transmission electron microscopy (TEM) combined with potassium permanganate staining to visualize where potassium permanganate stains electron-dense polymerized lignin (FIG. 1C,D, Supplemental FIG. 3) (Hepler, Fosket, and Newcomb 1970). Exodermal lignification occurs after endodermal CS deposition in the early maturation zone. First, lignification occurs in the corners of exodermal cells on their epidermal face, which we term the Stage 1 of exodermal lignification (FIG. 1B-D). In Stage 2 of exodermal differentiation, lignin levels increase at exodermal cell corners and lignin is deposited at the epidermal face of exodermal cells (FIG. 1B). Stage 3 of exodermal differentiation occurs in the middle of the root, where lignin content increases at the epidermal face of the exodermal cells and along the anticlinal cell walls towards the first inner cortical cell layer. Near the root-hypocotyl junction, Stage 4 of exodermal differentiation comprises maximal lignification of approximately 4 of the anticlinal cell wall (FIG. 1B). Polymerized lignin levels additionally increase at the epidermal face (FIG. 1B). This stage is maintained until the exodermis undergoes suberin deposition. This polar lignin cap in the exodermis in Solanum lycopersicum is also conserved in other Solanum species and Nicotiana benthamiana (Supplemental FIG. 1).

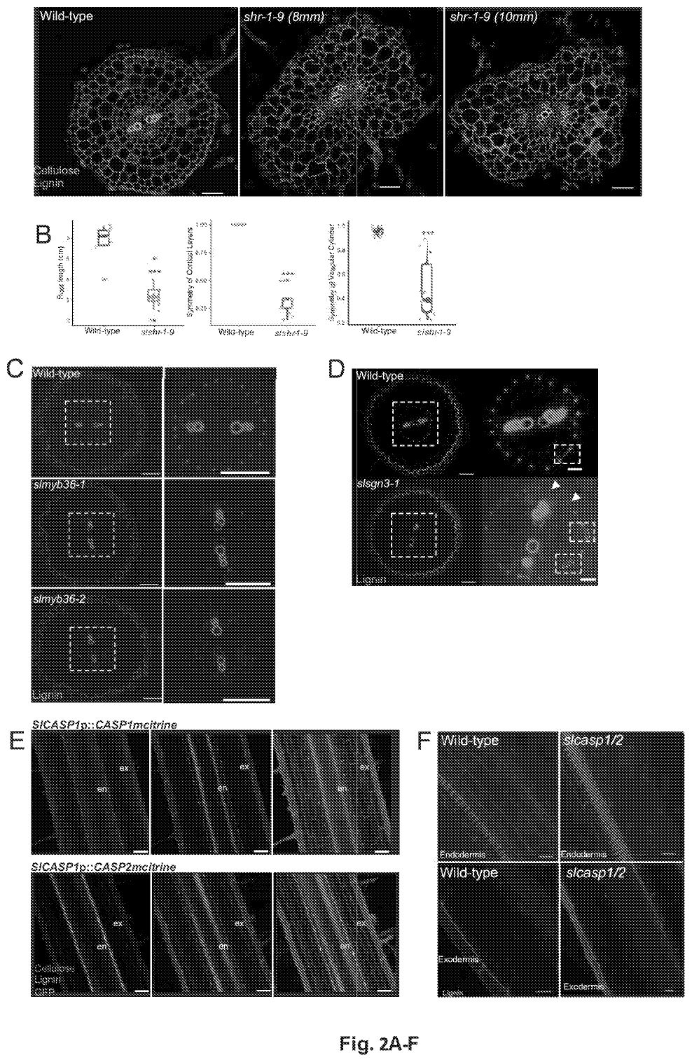

A hallmark of CS function is as an apoplastic barrier. To test if the polar exodermal lignin cap has an equivalent function, we used propidium iodide (PI) as an apoplastic tracer (Naseer et al. 2012; Wang et al. 2019). PI apoplastic transport was blocked by the exodermal cap after 30 minutes of incubation (FIG. 1E-F). Vascular PI presence is due to PI absorption within the meristem, where an exodermal lignin cap is not present and, to subsequent transport through the xylem. Piperonylic acid (PA), a monolignol biosynthetic inhibitor, was further used to demonstrate that exodermal barrier function is dependent upon lignin biosynthesis (Schalk et al. 1998; Naseer et al. 2012). Twenty-four hours of 200 μM PA exposure does not interfere with root growth but does disrupt root lignin biosynthesis within the endodermis and exodermis (Supplemental FIG. 2A-B). In PA-treated plants, Pt was observed after exposure within cortex cells (FIG. 1E). In addition, the PI signal in the exodermal lignin cap was more intense in control relative to PA-treated plants (FIG. 1F). Dependence of exodermal barrier function on lignin biosynthesis was further determined by complementation of the PA inhibitor-induced defects by the addition of two components of angiosperm lignin, the monolignols coniferyl-, and sinapyl alcohol (20 μM each), as previously used to demonstrate CS barrier function in Arabidopsis (Naseer et al. 2012). Treatment with both PA and monolignols restored lignin levels in the exodermis, increased lignin levels within the epidermis, and blocked PI transport at the epidermis (FIG. 1G-H).

Exodermal Differentiation is Genetically Distinct from the Endodermis

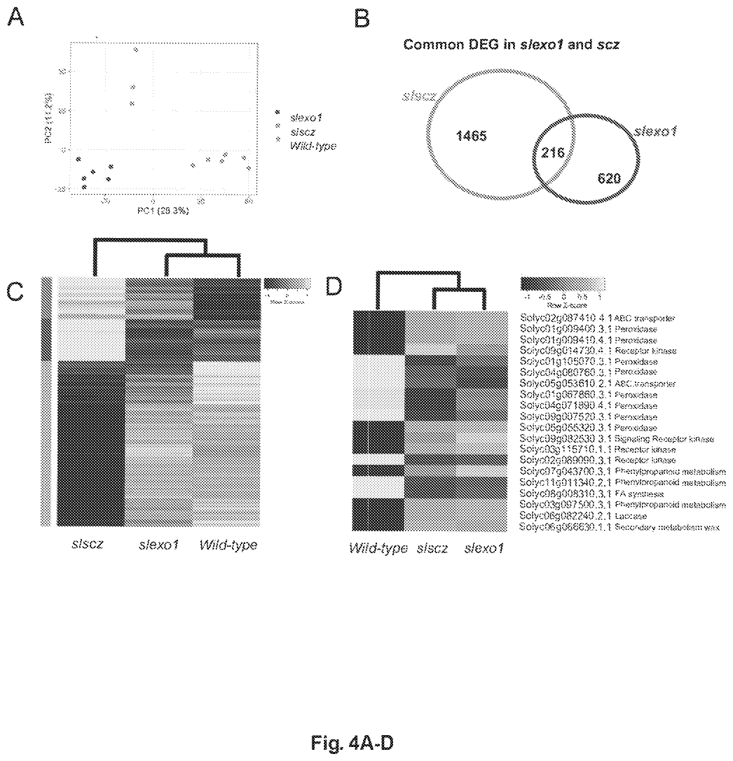

The tomato exodermis forms an apoplastic barrier with a distinct structure as compared to the endodermal Casparian strip, albeit, with a similar function. One working hypothesis is that genes that regulate endodermal differentiation also regulate exodermal differentiation. Alternatively, a distinct set of genes regulate this process. Recent evidence in maize demonstrates that subtle changes in the SHR regulator result in cortex layer multiplication and likely cortex specialization (Ortiz-Ramirez et al. 2021). The tomato exodermal layer results from asymmetric divisions of the cortex-endodermal stem cell niche (Ron et al. 2013). We, therefore, tested whether SHR regulates the formation of the exodermal polar lignin cap. SISHR (Solyc02g092370) transcript is produced in the stele, its protein is found in the endodermis, and SlSHR controls transcription of its downstream target SlSCR (Solyc10g074680) in Rhizobium rhizogenes-transformed roots (hairy roots; (Ron et al. 2014). Both R. rhizogenes and A. tumefaciens-generated (stably transformed roots) SlSHR CRISPR-Cas9 mutant alleles have defects in endodermal specification and differentiation, including the absence of the endodermal layer as well as the Casparian Strip (FIG. 2A, Supplementary FIG. 4A-B). In parts of the root where other cell types have already differentiated (exodermal lignin cap deposition and xylem secondary cell wall thickening), the mutant layer that surrounds the vasculature has no CS or ectopic lignin. More mature cells in this layer deposit ectopic lignin (FIG. 2A). Both slshr-1-9 and slshr-2 alleles additionally have asymmetric radial patterning in the ground tissue layers and the vascular cylinder (FIG. 2B, Supplementary FIG. 4C-D). Both alleles also develop shorter roots (FIG. 2B, Supplementary 4G). Phenotypes corresponding to a missing endodermal layer and a properly positioned and polymerized Casparian strip demonstrate that SISHR function is largely conserved between Arabidopsis and tomato for endodermal patterning and differentiation. A deviation from the Arabidopsis mutant phenotype is the radial asymmetry in the cortex cell layers (including the exodermis), and the ectopic lignin deposition in the mutant layer (FIG. 2A-B). However, all slshr alleles had wild type exodermal lignin cap (FIG. 2A).

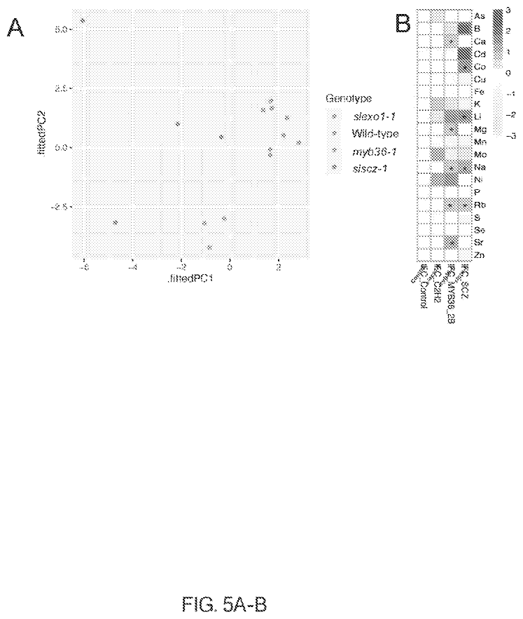

Barrier function of the exodermal polar cap is dependent on lignin biosynthesis. Loss of function Atmyb36 mutants lack the CS and present ectopic lignification in endodermal cells (Kamiya et al. 2015; Reyt et al. 2021). Phylogenetic analysis revealed 2 possible tomato MYB36 homologs of Arabidopsis MYB36 (AtSg57620)—Solyc07g&06750 and Solyc04g077260 (Supplemental FIG. 5A-B). Solyc07g006750 is the most closely phylogenetically related to AtMY136 (Supplemental FIG. 5B) and its ribosome-associated transcript abundance is enriched in the endodermis (Kajala et al. 2021) (Supplemental FIG. 5C). Solyc04g077260 is a recently reported homolog of AtMYB36 (P. Li et al. 2018) (Supplemental FIG. 5A). CRISPR-Cas9 hairy root mutants revealed that Solyc07g006750 (herein SlMYB36) but not Solyc04g077260 (herein SlMYB36-b) lack a CS (Supplemental FIG. 5D). A. tumefaciens-transformed CRISPR-Cas9 edited slmyb36 mutant alleles lack a CS but have no changes in ground tissue radial patterning. In contrast to atmyb36 mutants (Reyt et al. 2021; Kamiya et al. 2015), slmyb36 mutants have minimal ectopic lignification within the endodermis. Neither slmyb36 nor slmyb36-b mutants showed defects in the exodermal lignin cap (FIG. 2C, Supplemental FIG. 5D).

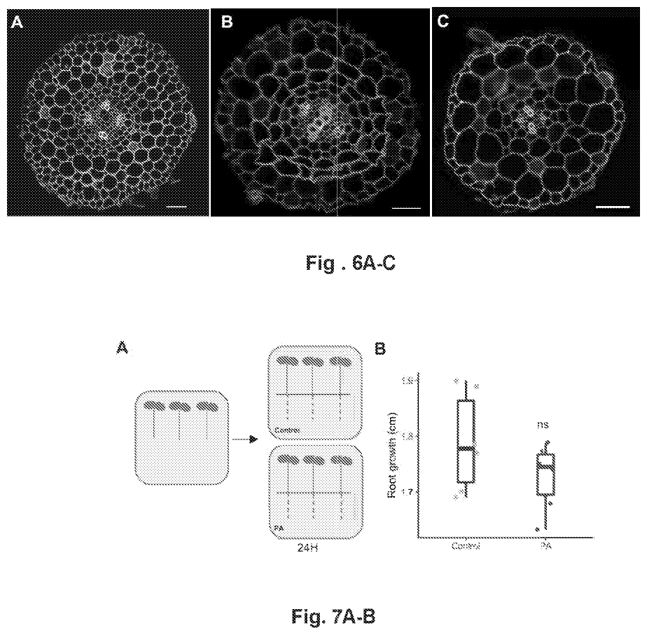

CASP proteins act as a scaffold to guide lignin biosynthesis enzymes to the CS domain (Roppolo et al. 2011). We identified 4 putative tomato AtCASP homologs (Solyc02g088160, Solyc06g74230, Solyc09g010200, Solyc10g083250) (Supplemental FIG. 6). Three of these have the extracellular loop1 (EL1) protein domain that is conserved in spermatophytes (Solyc06g074230, Solyc09g010200, and Solyc10g083250) (Roppolo et al. 2014) and were chosen for further study. Transcriptional GFP reporters demonstrated that SlCASP1 (Solyc06g074230) is expressed primarily in the endodermis with some expression in inner cortical and exodermal cells, SlCASP2 (Solyc1g083250) is primarily expressed in the endodermis and SlCASP3 (Solyc09g010200) is expressed in the endodermis, cortex, and exodermis (Supplemental FIG. 4E). SlCASP1 and SlCASP2 translational mCitrine reporters demonstrated that SlCASP1 and SlCASP2, and not the SlCASP3 proteins localize to the endodermal CS domain and not at the exodermal lignin cap domain in both hairy roots and stable transformed roots (FIG. 2E, Supplemental FIG. 4E). Hairy and stably transformed roots with CRISPR-Cas9 edits in both SlCASP1 and SlCASP2 genes (slcasp1slcasp2) showed no phenotype in the endodermal CS or the exodermal polar lignin cap (FIG. 2F).

The SGN3 receptor kinase along with the CIF peptide, controls the CS integrity surveillance system (Doblas et al. 2017; Nakayama et al. 2017). Phylogenetic analyses revealed one close homolog to AtSGN3/At4g20140, the Solyc05g007230 gene, and one to the AtCIF1/At2g16385 and AtCIF2/At4g34300 genes. Solyc01g109900 (also previously annotated as Solyc01g099895) (Supplemental FIG. 7 A-C). Transcriptional mCitrine reporters for SlSGN3 and SlCIF1 showed the SlSGN3 expression domain at the endodermis, inner cortex, and exodermis while the SlCIF1 expression domain is in the vasculature as in Arabidopsis (Doblas et al. 2017; Nakayama et al. 2017) (Supplemental FIG. 4F). Mutant alleles generated by stable and hairy root transformation via CRISPR-Cas9 editing of SlSGN3 revealed an interrupted CS as observed for Arabidopsis sgn3 (FIG. 2D; Supplemental FIG. 4H) (Pfister et al. 2014). The exodermal polar lignin cap is not affected in these mutants (FIG. 2D; Supplemental FIG. 411). These data collectively demonstrate that SlSHR, SlMYB36, SlCASP1, SlCASP2, and SlSGN3 are orthologs of Arabidopsis endodermal Casparian strip regulators, that endodermal differentiation is conserved between Arabidopsis and tomato and that tomato exodermal polar lignin cap differentiation is genetically distinct from endodermal differentiation,

Two Transcription Factors that Control Exodermal Differentiation

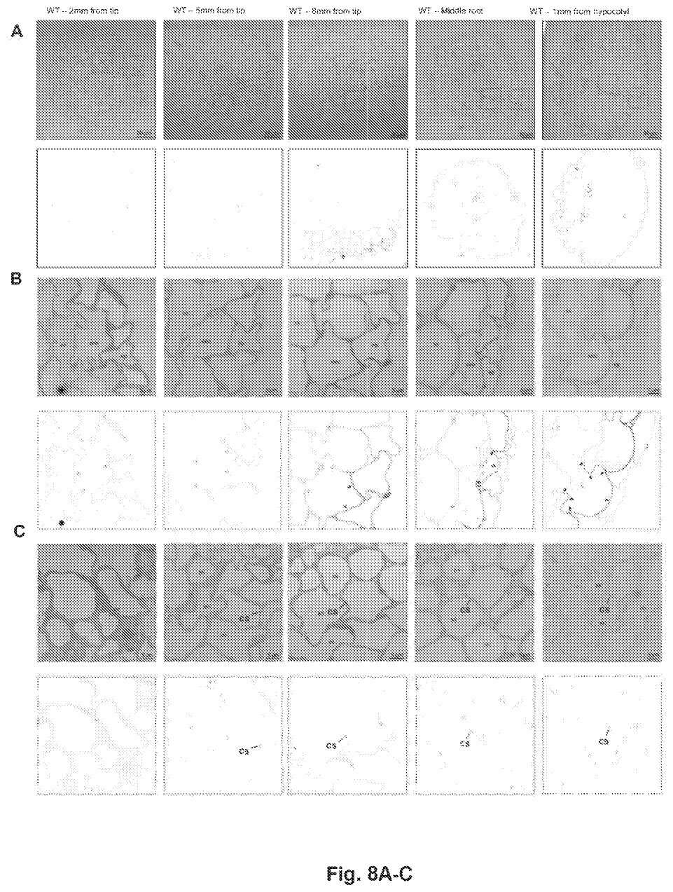

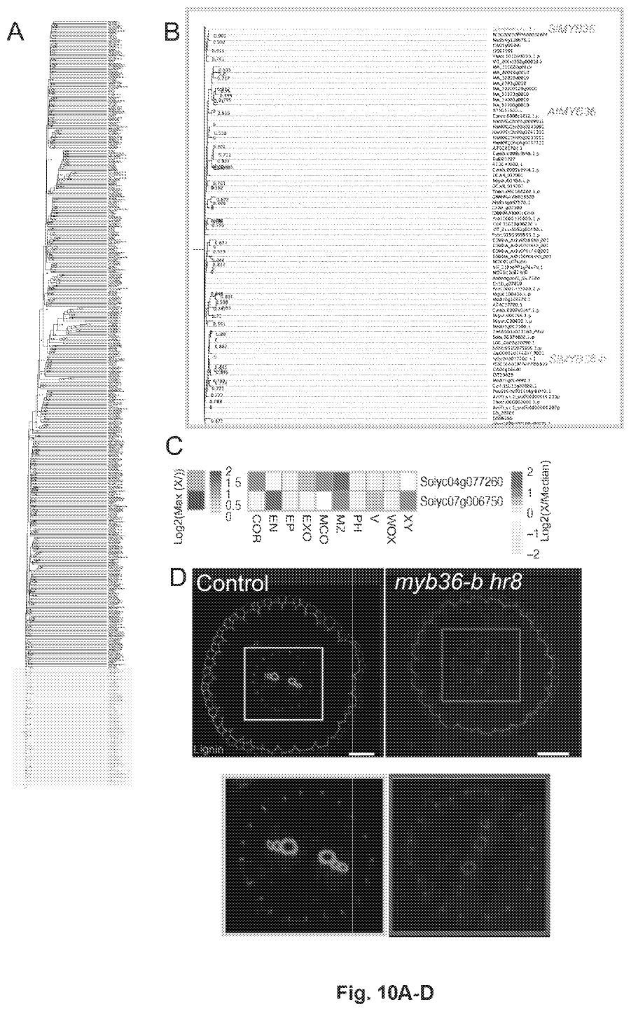

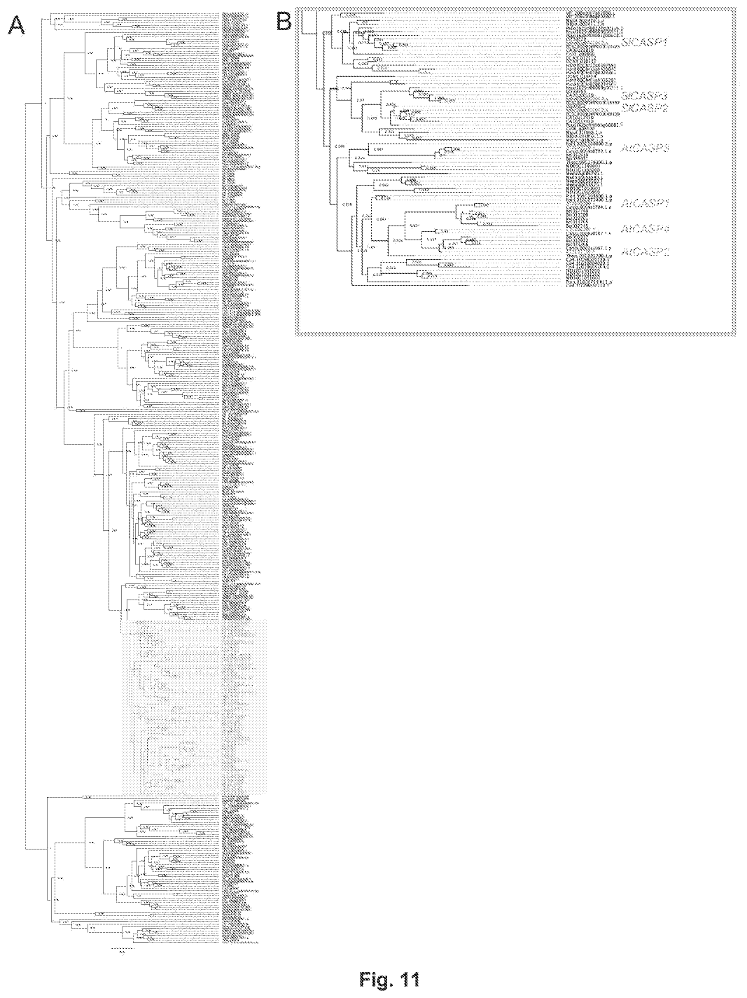

Mining of genes whose transcript abundance is enriched in the endodermis has been successful in identifying regulators of endodermal differentiation (Pfister et al. 2014; Y. Lee et al. 2013). We utilized this approach with the underlying hypothesis that transcription factors play a critical role in exodermal differentiation. We obtained a list of exodermal enriched ribosome-associated transcripts (Kajala et al. 2021) and identified transcription factors from multiple families (MYB, NAC, AP2/ERF, MADS-BOX, H D-Zip, WRKY, HLH, Zinc finger s, and HOMEOBOX) (Supplementary FIG. 8). Of these, MYB transcription factors were particularly attractive candidates given their function in endodermal barrier production, secondary cell wall biosynthesis and lignin deposition (Shukla et al. 2021; Kamiya et al. 2015; Miyamoto, Tobimatsu, and Umezawa 2020; J. Liu, Osbourn, and Ma 2015). We selected eight MYB family transcription factors for CRISPR-Cas9 mutagenesis and observed lignin production in wild type (R. rhizogenes-transformed with no binary plasmid) relative to independent mutant alleles of each transcription factor (Supplementary Table and Supplementary FIG. 10), We additionally mutated thirteen exodermal-enriched transcription factors (Table 1 and Supplementary FIG. 10). A lignin phenotype was only observed for a mutant of a zinc finger C2H2 family (Solyc09g01120) transcription factor (5 independent hairy root alleles) (Supplementary Table and Supplementary FIG. 11). These hairy root mutant alleles as well as a stably transformed mutant alleles of Solyc9g011120 (herein SlEXO1) had ectopic lignin deposition in the form of a polar lignin cap in inner cortex cells as well as a wild type exodermal polar cap (FIG. 3A, Supplementary FIG. 101). This phenotype suggests that SlEXO1 represses polar lignin cap formation in inner cortical layers and thus restricts barrier formation to the exodermal cells. The root length of slexo1-1 is additionally significantly shorter than wild type (FIG. 3F).

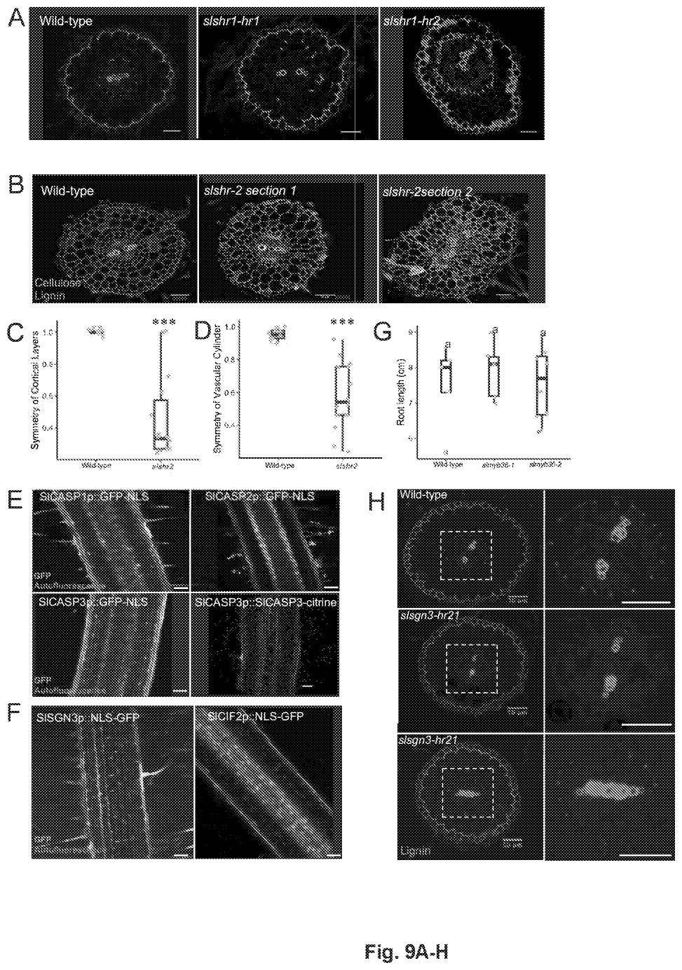

In Arabidopsis, SCHIZORIZA (AtSCZ/At1g46264), a heat shock transcription factor, regulates asymmetric stem cell divisions and specification of root cortex cell identity (ten Hove et al. 2010; Pernas, Ryan, and Dolan 2010). The exodermis is derived from the cortex-endodermis initial cell which gives rise to the endodermis, two inner cortical layers, and to the exodermis (an outer cortical layer) (Ron et al. 2013). As the exodermis is the outermost cortex cell layer in tomato, we hypothesized that a tomato homolog to AtSCZ could regulate exodermal differentiation. A single putative homolog of AtSCZ exists within the tomato genome, the Solyc04g078770 gene (herein SlSCZ) (Supplemental FIG. 9). A. tumefaciens-generated CRISPR-Cas9 mutant alleles have non-polar lignification (lignin coating all faces of the exodermal cell) in most, but not all exodermal cells (FIG. 3H). When lignin is non-polarized in the exodermal layer, lignin in the inner cortical layer is always polarly deposited (FIG. 3A). Moreover, some independent mutant alleles of slscz in hairy roots have groups of lignified cells where lignification occurs across multiple consecutive inner cortical layers, again with the inner-most cell containing polar lignification (Supplemental FIG. 11B-F). Sexually inherited mutations of SlSCZ (A. tumefaciens) are likely lethal as we were able to obtain only 2 independent mutant lines (FIG. 3A, Supplemental FIG. 11G). slscz-1 roots also have a short root phenotype, although much more extreme than slexo1-1—the root barely elongates after germination (FIG. 3G). Ectopic lignin is also present in the endodermis and the vascular cylinder is asymmetrically organized (FIG. 3A). Given the partial similarities in the slexo1-1 and slscz-1, we generated a double mutant to determine their genetic interaction, using the same guide RNAs to generate their respective mutations. Both hairy root and stable double mutant alleles showed a similar phenotype to the slscz-1 mutant, with ectopic lignin in exodermis and inner cortex along with a shorter root (FIG. 3A; Supplemental FIG. 11O).

To understand the location of these genes' transcripts and proteins, we created transcriptional and translational reporter lines. GFP driven by the SlEXO1 promoter was observed in the epidermis, exodermis, cortex and endodermis from the maturation zone but not in the meristem or elongation zone (FIG. 3B). The SLEXO1 protein was not detectable in hairy roots from a SlEXO1 translational reporter SlEXO1:SlEXO1-mcitrine both with and without the 5′ and 3′ untranslated regions (FIG. 3C). The SlSCZ transcriptional GFP reporter is expressed in the meristematic exodermis, cortex and endodermis and in the maturation zone in the epidermis, exodermis, cortex and endodermis. We could not detect GFP in the elongation zone (FIG. 3C). A translational reporter of SlSCZ fused to citrine showed that the SCZ protein is localized mainly at the meristematic exodermis, cortex and endodermis. The SCZ protein was not detected in the elongation or maturation zone (FIG. 3E). The differences between the expression of SlSCZ and the localization of the SlSCZ protein suggest the role of post-transcriptional modification in determining the final location of SlSCZ activity (J.-Y. Lee et al. 2006).

SCZ and SlEXO1 have Independent and Overlapping Downstream Regulation

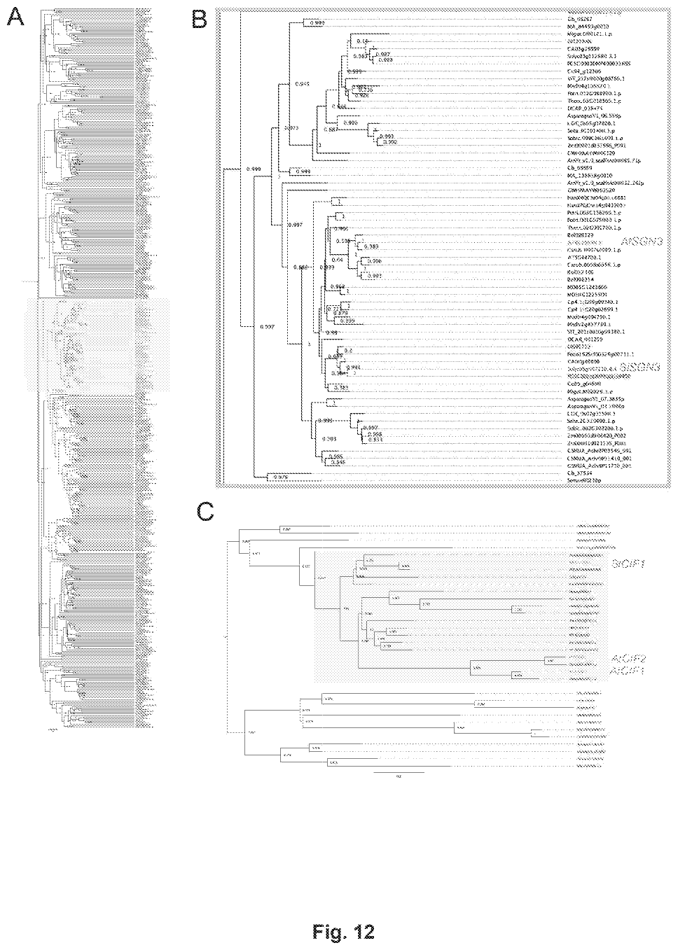

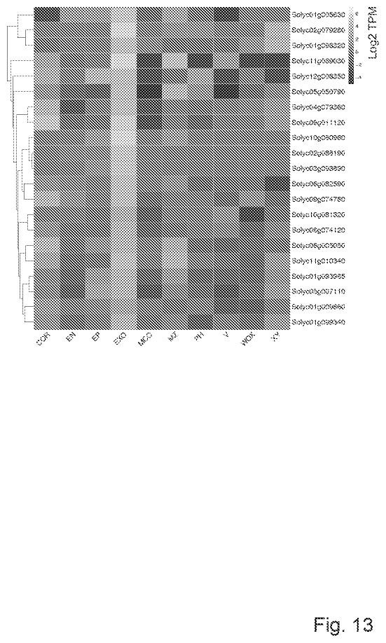

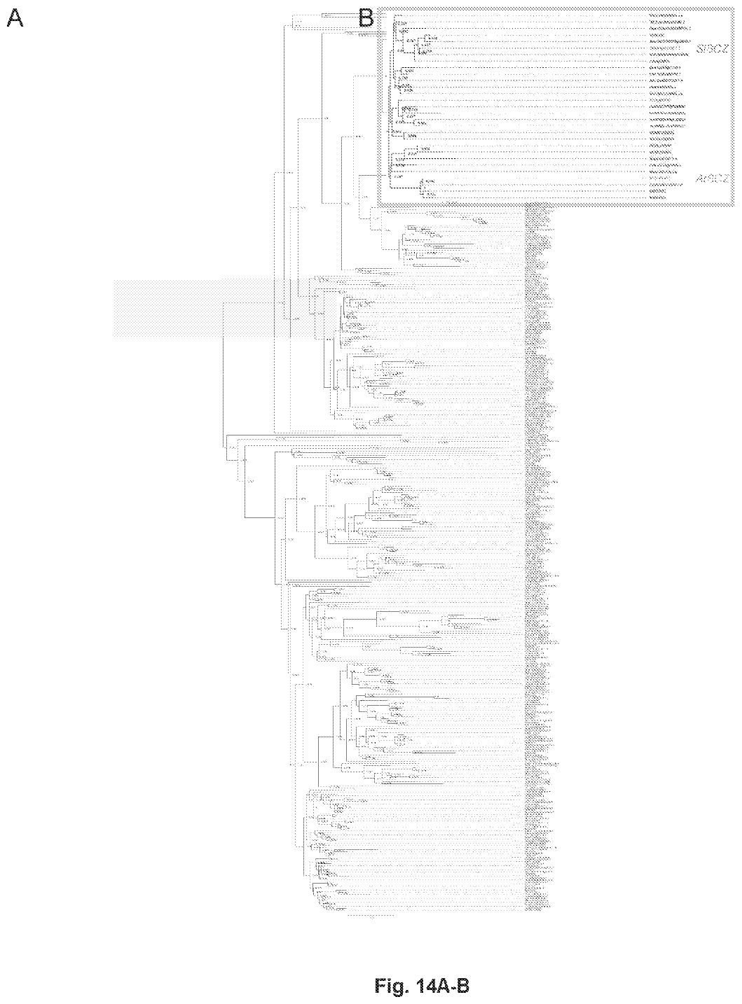

SlEXO1 and SlSCZ both repress lignification of inner cortical cell layers. Their double mutant phenotype further demonstrates that they genetically interact (FIG. 3A). These data suggest that SlEXO1 and SlSCZ may regulate common downstream regulatory pathways that control synthesis of the exodermal lignin barrier. To address this hypothesis, we conducted transcriptome profiling of two independent alleles each of slexo1 and slscz hairy root mutants. Principal component analysis revealed that the transcriptomes of each of these genotypes are transcriptionally distinct (FIG. 4A). Differentially expressed genes (DEGs) were identified for each mutant genotype relative to wild type (FDR test <0.05 FC>+/−1.3) with 263 genes representing common direct or indirect gene targets (FIG. 4B). Most of the slscz-differentially regulated genes were down-regulated relative to wild type (FIG. 4C). From the common DEGs (FIG. 4B) there is an enrichment in peroxidases (Fisher's exact test p-value=5.92e-05), genes related to phenylpropanoid metabolism and receptor like kinases (FIG. 4E). Gene Ontology categories associated with lignin, and secondary cell wall biosynthesis were up regulated in slexo1, consistent with the increased lignin in its inner cortical layer (FIG. 3A, Supplementary FIG. 12B). Conversely, genes associated with phenylpropanoid, and lignin biosynthesis were down-regulated in slscz, although genes associated with lignin catabolic processes, cell wall biogenesis and cell organization were up-regulated (Supplementary FIG. 12C). To confirm where these genes are expressed in wild type, we intersected these gene lists with those of tomato root cell types (Kajala et al., 2021). Genes differentially expressed in both the slexo1 and slscz mutant alleles have enriched transcript abundance within wild type inner cortex, exodermis, and epidermal translatomes (Supplementary FIG. 13A-B) and support the likely repressive function of these transcription factors.

### The Exodermal and Endodermal Barriers are Selective Checkpoints for Mineral Ions

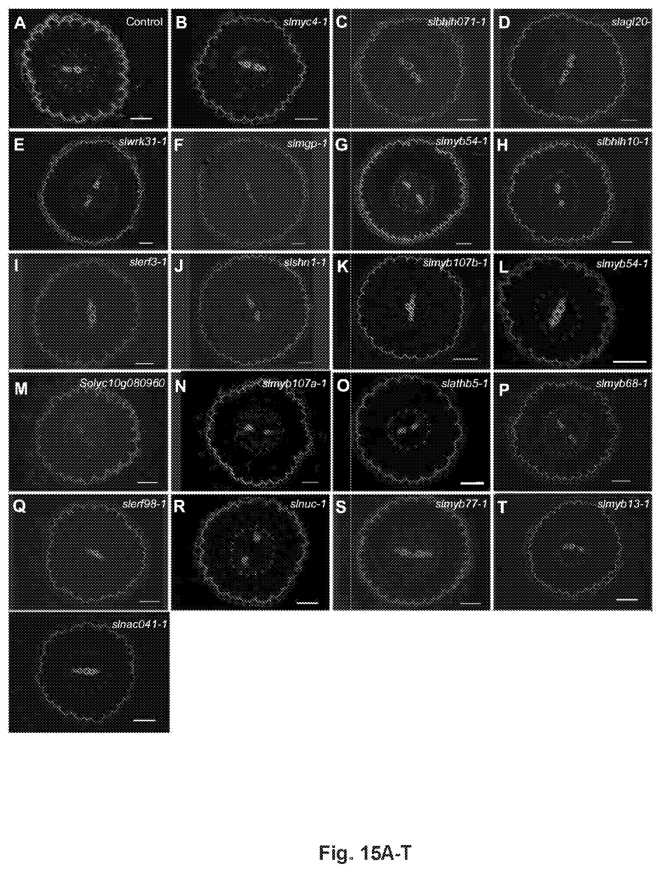

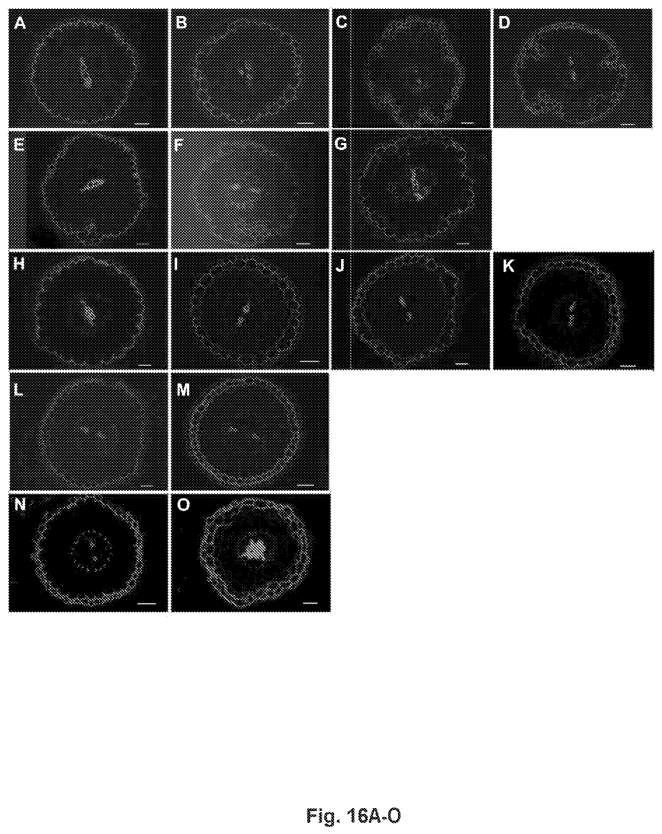

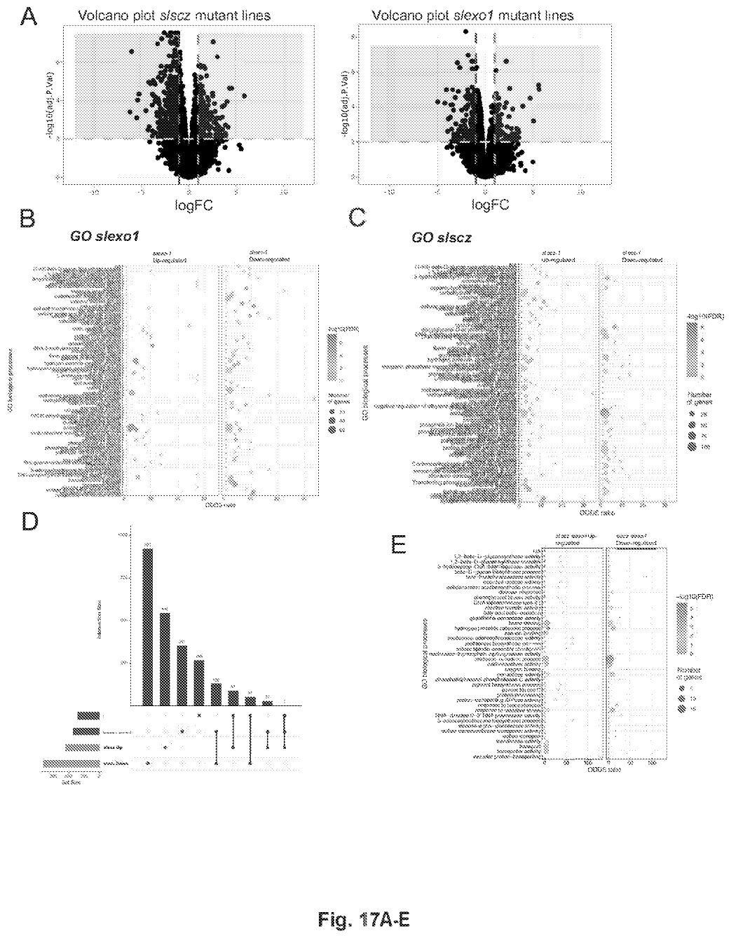

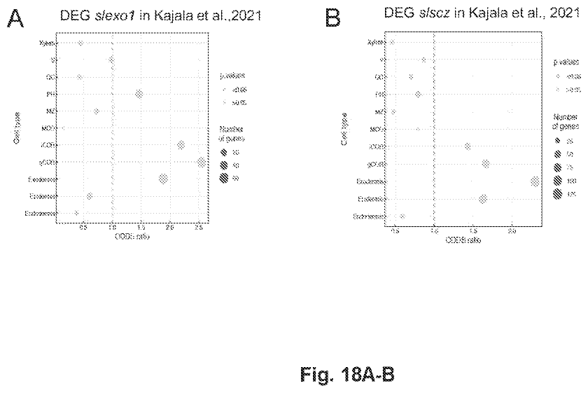

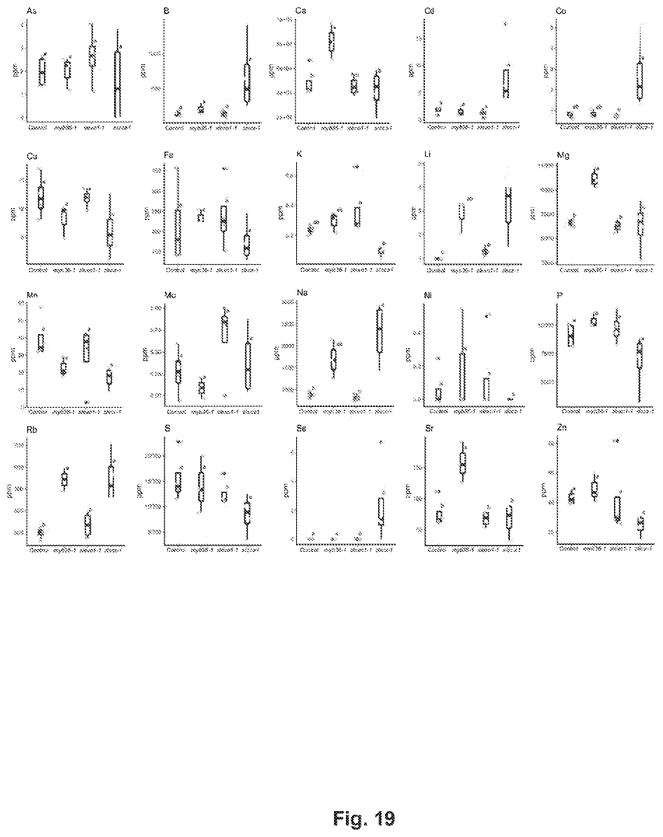

The endodermal Casparian strip plays a significant role in regulating the leaf ionome (Baxter et al. 2009; Hosmani et al. 2013; Pfister et al. 2014; Kamiya et al. 2015) consistent with the Casparian Strip as a selective barrier for mineral ion uptake to the shoot (Geldner 2013). The endodermis not only prevents mineral nutrients from entering the vasculature from the cortex but also prevents leakage back into the cortex of ions that are not translocated. In tomato, both the exodermis and endodermis have apoplastic barrier function. Are there specific ions that each barrier selectively controls? We tested this hypothesis by ionome profiling of the tomato shoot in the slmyb36-1 mutant (CS absence but a wild type exodermal lignin cap), the exo1-1 mutant (no defects in the CS and a polar lignin cap in the first inner cortical layer), and the slscz1-1 mutant (strong lignification in the exodermis and ectopic lignin in inner cortex cells and the endodermis). Leaves of four-week-old plants were analyzed for their elemental composition using inductively coupled plasma-mass spectrometry (ICP-MS) (FIG. 5). Principal component analysis (PCA) revealed that slmyb36-1 and slscz-1 have different ionome profiles than wild type, but the exo1-1 mutant ionome is more similar to wild type (FIG. 5A). The levels of all ions tested are not significantly perturbed in slexo1-1 relative to wild type (two-way ANOVA, p<0.05). Leaves of slmyb36-1 accumulate significantly increased sodium (Na), strontium (Sr), rubidium (Rb), calcium (Ca), magnesium (Mg) and lithium (Li) while molybdenum (Mo) is decreased in comparison with wild-type (FIG. 5B; Supplemental FIG. 14). The slscz-1 ionome is the most perturbed relative to wild type with increased accumulation of sodium (Na), rubidium (Rb), cobalt (Co), boron (B) and lithium (Li) and decreased levels of molybdenum (Mo), copper (Cu), manganese (Mn) and essential elements like potassium (K) and zinc (Zn) (FIG. 5B; Supplemental FIG. 14). Thus, the additional exodermal lignin barrier(s) in slexo1-1, slscz-1 and wild type do not completely compensate for the absence of the Casparian strip, and these barriers have unique and overlapping roles in selective mineral ion uptake.

### Discussion

The acquisition of multicellularity required the formation of intercellular barriers to facilitate communication and transport. The shape, composition and location of these barriers all inform their function. The best characterized of these barriers in plants is the endodermal Casparian strip, which is conserved across all angiosperms (Geldner 2013; Enstone, Peterson, and Ma 2002; C. Meyer and Peterson 2013; van Fleet 1961). As defined by Caspary and refined by others, this structure is formed of polymerized lignin that has impregnated the primary cell wall, as a belt in the center of the transversal and anticlinal walls (Alassimone, Naseer, and Geldner 2010; Enstone, Peterson, and Ma 2002; Naseer et al. 2012)(Caspary R. 1858; Caspary R. 1865). It fills up the entire space between two adjacent cells, including the middle lamellae, which means that the two cells are essentially fused with each other (Geldner 2013; Bonnett 1968; Haas and Carothers 1975; Karahara and Shibaoka 1992; Roppolo et al. 2011). The apoplastic barrier function of the Casparian strip is dependent upon its lignin composition (Naseer et al. 2012). In comparison, the root exodermis additionally forms apoplastic barriers that have been characterized for their functionality in many species (Peterson, Peterson, and Robards 1978; Peterson, Emanuel, and Wilson 1982; Peterson 1987; Lehmann et al. 2000). The shape and position of these barriers have also been elegantly described using histochemical stains and anatomical analyses, although many of these stains have not been able to distinguish between polymerized lignin and aliphatic suberin, nor do they facilitate ultrastructural observation (Enstone, Peterson, and Ma 2002) (Meyer and Peterson 2013). Here, we describe the developmental timeline by which a polymerized lignin cap seals the exodermal intercellular and epidermal-exodermal apoplastic space in a polar fashion. This structure does not appear to impregnate the primary cell wall, nor is it centrally localized, as is the case for the Casparian strip, although it does serve as an apoplastic barrier dependent on its lignin composition. How common is an exodermal barrier of this shape and composition across angiosperms? A similar polar structure composed of lignin and/or suberin was described in the literature for the seedless vascular plant Selaginella selaginoides and for Vinca minor, the former of which is recalcitrant to cell wall digestion and thus fuses exodermal cells together (Damus et al. 1997; Perumalla, Peterson, and Enstone 1990). Barriers of other shapes have been described. The outer exodermis in Iris germanica has a reverse structure to the polar cap (called a Y-shape), while Trillium grandiflorium, sugarcane, onion and barley roots have barriers sealing only the intercellular space of exodermal cells which resemble elongated Casparian strips (Lehmann et al. 2000; C. J. Meyer, Peterson, and Bernards 2011; Perumalla, Peterson, and Enstone 1990). Despite these differences in morphology, these structures appear to all act as apoplastic barriers, suggesting convergence.

The endodermis and exodermis have been described as twins for this convergence in composition and apoplastic barrier function ((Kajala et al. 2021) Canto-Pastor et al. submitted) (Geldner 2013). Two hypotheses can be formulated as to the mechanisms that give rise to the similarities in this stage of exodermal and endodermal differentiation—that they are governed by similar developmental factors or that they are determined by distinct regulatory mechanisms. Analysis of tomato SlSHR, SlMYB36, SlSGN3 and SIC ASP mutants demonstrates that these genes are conserved regulators of endodermis specification and differentiation, albeit with some differences. Mutation of tomato slshr reduces the number of ground tissue layers, and in the majority of the root, a Casparian strip is not evident. In the upper part of the root, however, a disorganized Casparian strip-like structure and ectopic lignin are observed in the mutant layer (the presumed second inner cortex layer), although there are no molecular markers for the second inner cortex layer. Furthermore, radial symmetry is perturbed in the ground tissue as well as the vascular cylinder. These additional phenotypes could reflect expanded function of SHR and modifications in its regulatory network in other dicot species. The slmyb36 mutant has a complete absence of lignin (FIG. 2C), while the atmyb36 mutant has ectopic lignin at cell corners which function as a compensatory barrier. The phenotype observed in tomato could only be achieved in Arabidopsis when both AtMYB36 and AtSGN3 genes were mutated (Reyt et al. 2021). A mutant phenotype was observed when the tomato slsgn3 was mutated (FIG. 2D), suggesting that a surveillance integrity mechanism does exist in tomato, although it is not necessary to compensate for a lack of endodermal Casparian Strip. It is possible that this mechanism is not necessary in tomato as there is a cell type with an additional apoplastic barrier. Whether this remains true for other plant species with an exodermis remains to be seen. These data demonstrate that distinct regulatory mechanisms from the endodermis determine exodermal specification and deposition of the polar cap. The question of if other species with an exodermal barrier have co-opted the SHR/MYB36/SGN3 molecular regulators is still open.

Our extensive genetic screen for exodermal lignin cap regulators identified two transcription factors that repress lignin cap deposition in the inner cortex layer. with SlSCZ additionally regulating the polar deposition of lignin in the exodermis. The AtSCZ gene was previously identified as a regulator of cortex cell identity in Arabidopsis (ten Hove et al. 2010; Pernas, Ryan, and Dolan 2010). As there are multiple cortex cell layers (including the exodermis) in tomato, it is possible that SlSCZ has evolved to participate specifically during exodermis specification/differentiation, although defects in epidermal morphology are observed (FIG. 3A). SlSCZ and SlEXO1 reporter localization suggest that SlSCZ may regulate exodermal/inner cortical specification and differentiation, while SlEXO1 regulates differentiation only. Collectively these data demonstrate spatial restriction of exodermal differentiation to the outermost cortex cell layer. Plant species with a multiseriate (multiple layered) exodermis (Peterson and Perumalla 1990) resemble these mutant phenotypes. Links with the environment and these barriers have been observed (C. J. Meyer, Seago, and Peterson 2009; C. J. Meyer, Peterson, and Steudle 2011; X. Li 2018) and it is intriguing to speculate that SlSCZ and SlEXO1 enable this flexibility.

The relationship between these two factors can be genetically inferred from the slscrslexo1 double mutant. The exodermal layer is largely fully lignified and when this occurs, the inner cortical layer has a polar lignin cap. The vascular cylinder is also elongated, and epidermal cells are misshapen. We propose two hypotheses regarding SCZ and EXO1. The double mutant phenotype is similar to that of slscz with respect to the complete lignification of the exodermal layer. One model is that the scz phenotype is epistatic to that of slexo1, and SCZ is genetically downstream of SlEXO1. However. slscz and slexo1 phenotypes are not opposing, as is typical of developmental genetic analysis predicting epistasis. An equally plausible model is that these genes act additively, or in parallel pathways. Given the nature of these phenotypes, it is difficult to distinguish which model is best. Despite these genes both being transcription factors, no changes in gene expression of the other factor were observed in their respective loss-of-function mutant alleles, although these data were not collected at cell type resolution. This suggests that the relationship between these factors is not via transcriptional regulation. Asymmetry in the vascular cylinder of both slshr and slscz was also observed, suggesting these two factors could coordinately control aspects of vascular development. We utilized cell type-enriched gene lists to identify candidate genes that control exodermis development and to determine the domain of function of these genes, under the assumption their function is cell autonomous. These phenotypes in the vasculature (slscr, slshr and slscnslexo1) as well as the epidermis (slscz, slscrexo1) raise the question of whether cell non-autonomous signals may further contribute to specification of the positioning of the polar cap. Additional clues to the factors that contribute to the positioning and polymerization of the cap were identified in loss-of-function mutant alleles of both transcription factors. These include peroxidases and a laccase that could contribute to lignin polymerization as well as many signaling proteins including kinases and leucine rich receptor kinases.

The tomato exodermal and endodermal barriers do act as a selective barrier for mineral ion uptake as previously observed (C. J. Meyer, Peterson, and Steudle 2011). No significant changes in element uptake were observed in the slexo1-1 mutant, suggesting that although an internal exodermal lignin cap is observed, it is not an additional functional barrier (FIG. 5A,B). The ionome of slmyh36-1 and slscz-1 were quite different from wild type and each other (FIG. 5A) demonstrating extensive differences in ion uptake in a genotype with an absent Casparian Strip relative to that of a genotype with multiple lignified cortical layers. An increase in Cadmium (Cd), Cobalt (Co) and Lithium (Li) were solely observed in slscz-1, suggesting that specific transporters may be enriched in cortical cells with this barrier type (FIG. 5B). Calcium (Ca), magnesium (Mg) and strontium (Sr) were enriched in the ionomes of slmyb36, demonstrating that the Casparian Strip likely restricts their passage to the vasculature (FIG. 5B) and the lack of compensation by either slexo1-1 or slscz-1 indicates their inability to compensate for this missing barrier. This is in contrast to observations for silicon and manganes uptake in rice where their transporters are expressed in both the exodermis and the endodermis (Ma et al. 2007)(Sasaki et al. 2012). Both sodium (Na) and rubidium (Rb) were upregulated in slmyb36-1 and slscz-1. Perhaps this could be explained by the Casparian strip restricting their uptake, and the extra barriers in slscz-1 promoting their uptake. The function of these barriers may additionally have an influence in response to the abiotic and biotic environment. In the slmyb36 mutant there are no changes in essential elements as was observed in atmyb36 (Reyt et a. 2021). A mutant with complete absence of the exodermal barrier remains to be identified and is necessary to completely delineate the roles of each of these barriers. A breeding advantage may be facilitated by the consecutive lignin caps of slexo1-1 (exodermis and inner cortex) in toleration of higher ion concentrations in the soil matrix or rhizosphere as results from a restriction in water availability or high salinity as was reported for a species with a multilayered exodermis (C. J. Meyer, Peterson, and Steudle 2011). In addition, the presence or absence of the exodermal lignin cap and the endodermal Casparian Strip may influence uptake of specific microbes or other biotic organisms which could be selectively modulated for increased pathogen resistance (Kawa and Brady 2022). We also hypothesize that the differences between the two exodermis mutants ion phenotypes could be explained not just by the presence or absence of ectopic lignin in exodermis and inner cortex, but also by the amount and composition of lignin in these cell types, as was proposed for corner lignification in the atmyb36 mutant (Reyt et al. 2021). Finally, these distinct barriers may provide mechanical support to the root system as it grows through the soil, as described for lignin in other plant tissues (Polo et al. 2020; Q. Liu, Luo, and Zheng 2018; Zhao et al. 2020; Emonet and Hay 2022).

To conclude, we have described a polarly localized lignin barrier in the tomato root exodermis which functions as an apoplastic barrier and controls selective mineral ion uptake. We have elucidated a gene set that controls its developmental regulation, and which is distinct from endodermal regulation. Transcriptomic analyses obtained of their mutants identified several factors that may play a role in positioning and polymerization of the lignin cap.

### Illustrative Materials and Methods

### Plant Material and Growth Conditions

S. lycopersicum (cv. M82, LA3475) seeds were surface sterilized with 70% ethanol for 5 minutes and then treated with commercial bleach (70%) for 20 minutes. Seeds were subsequently washed three times with deionized water. Seeds were transferred to 12 cm×12 cm plates containing 4.3 g/L Murashige and Skoog medium (Caisson), 0.5 g/l MES pH 5.8 and 10 g/L agar (Difco). Plates were placed in a 23° C. growth chamber for seven to 10 days with 16 hours of light and 8 hours of dark per day. All sections are from 5-6 days-old plants.

### Molecular Cloning

For the transcriptional constructs a region of 2-3 Kb upstream the ATG was selected as the promoter region. This PCR fragment was cloned into D-TOPO (Fisher scientific Cat no 450218). The D-TOPO cloned with the promoter fragment was recombined using an LR reaction into the vector pMK7FNFm14GW (Karimi et al. 2007)

For the translational constructs a region of 2-3 Kb upstream the ATG was selected as the promoter region. This fragment was cloned in 5 primeTOPO (Thermo Fisher scientific Cat no 591-20), the CDS was cloned in D-TOPO (Fisher scientific Cat no 450218) and the mcitrine cloned in P2P3 gateway vector was kindly provided by the lab of Niko Geldner. All three vectors were recombined by multiple LR in the destination vector pB7m34GW (Karimi, De Meyer, and Hilson 2005).

Rhizobium rhizogenes Transformation

“Hairy root” transformation with R. rhizogenes (ATCC 15834) was conducted according to (Ron et al. 2014). Cotyledons from seven to ten day old plants were cut and immersed immediately in a suspension of R. rhizogenes containing the desired binary vector. Cotyledons were then floated on sterile Whatman filter paper and co-cultivated in the dark on MS agar plates (1× vitamins, 3% sucrose, 1% agar) for three days at 25° C. in the dark, without antibiotic selection. Cotyledons were then transferred to a selective plate (MS with cefotaxime; 200 mg/L; 100 mg/L kanamycin). 30 independent antibiotic-resistant roots were subcloned for each transgenic line for future analyses with Cefotaxime+Kanamyzin for 2 rounds of subcloning and maintained in media with Cefotaxime after that period.

Agrobacterium tumefaciens Transformation

The UC Davis Plant Transformation Facility generated transgenics using A. tumefaciens and tissue-culture based protocols.

### Histochemistry and Imaging

Hairy roots of transcriptional fusions (SlCASPs, SlSGN3, SKC7 SIXO1 and SlSCZ) were imaged on a Zeiss LSM700 confocal (water immersion, ×20 objective) with excitation at 488 nm and emission at 493-550 nm for GFP and mcitrine and excitation at 555 nm and emission at 560-800 nm for autofluorescence. For the SlSCZ:GFP transcriptional fusion and the SlSCZ-mcitrine translational fusion images within the meristematic zone, hairy roots were cleared in Clearsee buffer for 2 weeks (Ursache et al. 2018), stained with calcofluor white for 30 minutes, washed two times and visualized using Confocal Laser Scanning Microscopy with a Zeiss Observer Z1. The same protocol was used for the images of the stable lines of the translational fusion constructs (SlCASP1:SlCASP1::mCitrine and SlCASP2:SlCASP2::mcitrine) and the slcasp1slcasp2 stable CRISPR mutant.

For root sections stained with basic fuchsin (Fisher scientific Cat no 632-99-5) and calcofluor white (PhytoTechnology laboratories Cat no 4404-43-7), 1 cm segments from the root tip were embedded in 3% agarose and were sectioned at 150-200 μM using a vibratome (Leica VT1000 S). The sections were stained in Clearsee buffer (Ursache et al. 2018) with basic fuchsin for 30 minutes then washed two times. In some cases, the sections were stained with calcofluor after basic fuchsin for 30 minutes and washed two times. Confocal Laser Scanning microscopy was performed on a Zeiss LSM700 confocal with the 20× objective, basic fuchsin: 550-561 nm excitation and 570-650 nm detection. Root samples were mounted in ClearSee (Ursache et al., 2018) and scanned and imaged on a Zeiss Observer Z1 with the 20× or 40× objective, basic fuchsin: 550-561 nm excitation and 570-650 nm detection and calcofluor white: 405 nm excitation and 425-475 nm detection.

### ICP-MS

Seeds from myb36-1, slexo1-1. slscz-1 and wild-type M82 were germinated in 1% MS+1% sucrose square plates and then 6 days after germination they were transferred to pots in soil. Four plants per genotype were randomized on a tray and watered two times a week with fertilized water for one month. The same portion of the compound leaf was collected from each plant and dried in a falcon tube in a 60 C oven for 12 hours. Dried tissue was homogenized with a mortar and pestle and the dried powder was weighed for further analyses. The powder was digested in concentrated nitric acid for 3 hours at 100 C. After digestion, nitric acid was evaporated at 80-100 C. The same volume of 2% Nitric acid was added to each sample. The standards for the 20 elements were from Sigma Aldrich (01969-100ML-F, 01932-100ML, 19051-100ML-F,36379-100ML-F,30329-100ML-F,68921-100ML-F,06335-100ML,12292-100ML,30083-100ML-F,74128-100ML,68780-100ML,00462-100ML,28944-100ML-F,38338-100ML,01444-100ML, 18021-100ML, 50002-100ML, 75267-100ML) and Fisher scientific (CLZN22M, CLFE2-2Y) and were prepared in a dilution series. Samples were analyzed in a Perkin Elmer ICP-MS. Calibration curves were made using the standards and the amount (part per million) was calculated based on the calibration curve. Data analyses were performed in R for the PCA plot (prcomp package) and the heatmap (pheatmap from Biostrings package). A two-way ANOVA statistical test was performed (p-value<0.05) to identify elements significantly different from wild type. The relative amount for each element per genotype in the Heatmap was calculated as the log 2 fold change from the average of 3-4 replicates.

### Apoplastic Tracer Assays

Seeds of M82 were grown in 1% MS+1% sucrose. Four days after germination, plants were transferred to new MS square plates with/without 200 μM of piperonylic acid (Sigma P49805-5G) and 200 μM piperonylic acid plus 10 mM monolignols (Sigma Aldrich 223735-100MG,404586-100MG) for 24 hours in the dark, with DMSO as the solvent. Three cm segments from the root tip including the root meristem were exposed to 15 μg/mL PI (P4170; Sigma) in the dark at 30° C. for 1 h. The roots were washed in water and one cm segments from the root tip were embedded in 3% agarose and made sections of 150-200 μM using a vibratome (Leica VT1000 S). Confocal Laser Scanning microscopy was performed on a Zeiss LSM700 confocal with the 20× objective, at 405 nm excitation and 600-650 nm detection.

### Transmission Electron Microscopy

Tomato roots were fixed in 2.5% glutaraldehyde solution (EMS, Hatfield, PA) in phosphate buffer (PB 0.1 M [pH 7.4]) for 1 hour at room temperature and subsequently fixed in a fresh mixture of osmium tetroxide 1% (EMS) with 1.5% potassium ferrocyanide (Sigma, St. Louis, MO) in PB buffer for 1 hour at room temperature. The samples were then washed twice in distilled water and dehydrated in acetone solution (Sigma, St Louis, MO, US) in a concentration gradient (30% for 40 minutes; 50% for 40 minutes; 70% for 40 min and 100% for 1 hour 3 times. This was followed by infiltration in LR White resin (EMS, Hatfield, PA, US) in a concentration gradient (33% LR White 33% in acetone for 6 hours; 66% LR White in acetone for 6 hours; 100% LR White for 12 hours two times) and finally polymerized for 48 hours at 60° C. in an oven in atmospheric nitrogen. Ultrathin sections (50 nm) were cut transversely at 2, 5 and 8 mm from the root tip, the middle of the root and 1 mm below the hypocotyl-root junction, using a Leica Ultracut UC7 (Leica Mikrosysteme Gmbi, Vienna, Austria), picked up on a copper slot grid 2×1 mm (EMS, Hatfield, PA, US) and coated with a polystyrene film (Sigma, St Louis, MO, US).

Visualization of lignin deposition in the exodermis and the Casparian strip was performed using permanganate potassium (KMnO₄) staining (Hepler et al., 1970). The sections were post-stained using 1% KMnO₄ in H₂O (Sigma, St Louis, MO, US) for 45 minutes and rinsed several times with H₂O.

Micrographs and panoramic images were taken with a transmission electron microscope FEI CM100 (FEI, Eindhoven, The Netherlands) at an acceleration voltage of 80 kV with a TVIPS TemCamF416 digital camera (TVIPS GmbH, Gauting, Germany) using the software EM-MENU 4.0 (TVIPS GmbH, Gauting, Germany). Panoramic images were aligned with the software IMOD (Kremer et al, 1996)(Kremer, Mastronarde, and McIntosh 1996).

### Transcriptome Profiling and Data Analysis

For the RNA-seq analyses 3 cm of same developmental stage of hairy roots from skscz-hr5, slsc-hr12, slexo1-hr6, slexo1-hr7 and wild-type M82 transformed with R. rhizogenes with no vector in three biological replicates. RNA was extracted with the Direct-zol RNA MiniPrep Plus kit (Neta Scientific Cat no RPI-ZR2053). cDNA libraries were made with the QuantSeq 3′ mRNA-Seq Library Prep kit from Lexogen (Cat no 015 QuantSeq FWD 3′ mRNA-Seq Library Prep Kit—with single indexing). Sequences were pooled, trimmed and filtered using Trimmomatic (Bolger, Lohse, and Usadel 2014). Trimmed reads were pseudo-aligned to the ITAG4.1 transcriptome (cDNA) (Tomato Genome Consortium, 2012) using Kallisto (v0.43.1)(Bray et al. 2016), with the parameters -b 100-single -l 200-s 30, to obtain count estimates and transcript per million (TPM) values. Differentially expressed genes (DEGs) were detected with the limma R package, using normalized CPM values as required by the package (Ritchie et al. 2015). CPM values were normalized with the voom function (Law et al., 2014) using TMM normalization. The functions lmfit, contrasts.fit, and ebayes were used to fit a linear model and calculate differential gene expression between the different contrasts. Genes with a log 2 fold change (FC) value >0.6 and FDR adjusted P value (adj.P.Val)≤0.05 were considered as differentially expressed. The fdr method was used to control the false discovery rate (FDR) (Benjamini and Hochberg 1995).

### Generation of CRISPR-Cas9 Edited Constructs: Hairy Root Mutant Screen

Target guide RNAs were designed using the CRISPR-PLANT web tool (https www site genome.arizona.edu/crispr/CRISPRsearch.html). In cases where CRISPR-PLANT did not specify at least three guides with GC content between 40 to 60%, guides were designed with CRISPR-P V2 (http site //crispr.hzau.edu.cn/cgi-bin/CRISPR2/CRISPR), using the U6 snoRNA promoter with <3 mismatches within the target gene coding sequence. Genomic sequences (ITAG3.2) were retrieved from Phytozome (https site //phytozome-next.jgi.doe.gov/) and gene maps were constructed with SnapGene. Primers for genotyping were designed with Primer-BLAST software (https www site ncbi.nlm.nih.gov/tools/primer-blast/). Primer specificity was checked against the Solanum lycopersicum using blast in Phytozome. The guide RNA was cloned in a method adapted from Lowder et al. (2015). In summary, oligos containing the sgRNA PAM sequence were phosphorylated and ligated into pYPQ131-3 vectors and recombined into p278 via Gateway cloning. A p278 vector containing all 3 gRNA expression cassettes was then recombined by Gateway into a pMR286 vector containing Cas9 and Kan resistance expression cassettes (Bari et al. 2019). The final CRISPR vector was introduced into Rhizobium rhizogenes (hairy roots) and Agrobacterium tumefaciens (stable lines) to generate transgenics.

### Phylogenetic Tree Construction

The following pipeline from Kajala et al., 2021 was utilized. Forty two representative proteomes were downloaded from Phytozome, Ensembl, or consortia sites depending on availability. These include early-diverging taxa, and broadly representative taxa from angiosperms. Next. blastp (Madden, 2013) was used to identify homologous sequences within each proteome based on a sequence of interest, with options “-max target_seqs 15-evalue 10E-6-qcov hsp perc 0.5-outfnt 6.” To refine this set of sequences, a multiple sequence alignment was generated with MAFFT v7 (Katoh and Standley, 2013) (option-auto), trimmed with trimal (Capella-Gutiérrez et al., 2009) with setting “-gappyout,” and a draft tree was generated with FastTree (Price et al., 2010). A monophyletic subtree containing the relevant sequences of interest was selected and more distantly related sequences were removed from the list of sequences. Tree construction methodology was informed by Rokas, 2011. For the final trees, MAFFT v7 using L-INS-i strategy was used to generate a multiple sequence alignment. Next, trimal was used with the -gappyout option. To generate a phylogenetic tree using maximum likelihood, RAxML was used with the option -m PROTGAMMAAUTO and 100 bootstraps. Finally, bipartitions with bootstrap values less than 25% were collapsed using TreeCollapserCL4 (http://emmahodcroft.com/TreeCollapseCL.html). Resulting trees were rooted on sequences from the earliest-diverging species represented in the tree.

It is understood that the examples and embodiments described herein are for illustrative purposes only and that various modifications or changes in light thereof will be suggested to persons skilled in the art and are to be included within the spirit and purview of this application and scope of the appended claims.

All accession numbers, publications, patents, and patent applications cited herein are hereby incorporated by reference with respect to the material for which they are expressly cited.

### REFERENCES CITED BY AUTHOR/YEAR

Alassimone, J., S. Naseer, and N. Geldner. 2010. “A Developmental Framework for Endodermal Differentiation and Polarity.” Proceedings of the National Academy of Sciences. https://doi.org/10.1073/pnas.0910772107.
Barberon, Marie, Joop Engelbertus Martinus Vermeer, Damien De Bellis, Peng Wang, Sadaf Naseer, Tonni Grube Andersen, Bruno Martin Humbel, et al. 2016. “Adaptation of Root Function by Nutrient-Induced Plasticity of Endodermal Differentiation.” Cell 164 (3): 447-59.
Baxter, Ivan, Prashant S. Hosmani, Ana Rus, Brett Lahner, Justin O. Borevitz, Balasubramaniam Muthukumar, Michael V. Mickelbart, Lukas Schreiber, Rochus B. Franke, and David E. Salt. 2009. “Root Suberin Forms an Extracellular Barrier That Affects Water Relations and Mineral Nutrition in Arabidopsis.” PLoS Genetics 5 (5): e1000492.
Benfey, P. N., P. J. Linstead, K. Roberts, J. W. Schiefelbein, M. T. Hauser, and R. A. Aeschbacher. 1993. “Root Development in Arabidopsis: Four Mutants with Dramatically Altered Root Morphogenesis.” Development 119(1): 57-70.
Doblas, Verónica G., Elwira Smakowska-Luzan, Satoshi Fujita, Julien Alassimone, Marie Barberon, Mathias Madalinski, Youssef Belkhadir, and Niko Geldner. 2017. “Root Diffusion Barrier Control by a Vasculature-Derived Peptide Binding to the SGN3 Receptor.” Science. https://doi.org/10.1126/science.aaj1562.
Enstone, Daryl E., Carol A. Peterson, and Fengshan Ma. 2002. “Root Endodermis and Exodermis: Structure, Function, and Responses to the Environment.” Journal of Plant Growth Regulation. https://doi.org/10.1007/s00344-003-0002-2.
Gallagher, Kimberly L., Alice J. Paquette, Keiji Nakajima, and Philip N. Benfey. 2004. “Mechanisms Regulating SHORT-ROOT Intercellular Movement.” Current Biology: CB 14 (20): 1847-51.
Geldner, Niko. 2013. “The Endodermis.” Annual Review of Plant Biology. https://doi.org/10.1146/annurev-arplant-050312-120050.
Helariutta, Y., H. Fukaki, J. Wysocka-Diller, K. Nakajima, J. Jung, G. Sena, M. T. Hauser, and P. N. Benfey. 2000. “The SHORT-ROOT Gene Controls Radial Patterning of the Arabidopsis Root through Radial Signaling.” Cell 101 (5): 555-67.
Hepler, Peter K., Donald E. Fosket, and Eldon H. Newcomb. 1970. “LIGNIFICATION DURING SECONDARY WALL FORMATION IN COLEUS: AN ELECTRON MICROSCOPIC STUDY.” American Journal of Botany. https://doi.org/10.1002/j.1537-2197.1970.tb09793.x.
Hosmani, P. S., T. Kamiya, J. Danku, S. Naseer, N. Geldner, M. L. Guerinot, and D. E. Salt. 2013. “Dirigent Domain-Containing Protein Is Part of the Machinery Required for Formation of the Lignin-Based Casparian Strip in the Root.” Proceedings of the National Academy of Sciences. https://doi.org/10.1073/pnas.1308412110.
Hove, Colette A. ten, Viola Willemsen, Wouter J. de Vries, Anja van Dijken, Ben Scheres, and Renze Heidstra. 2010. “SCHIZORIZA Encodes a Nuclear Factor Regulating Asymmetry of Stem Cell Divisions in the Arabidopsis Root.” Current Biology: CB 20 (5): 452-57.
Kajala, Kaisa, Mona Gouran, Lidor Shaar-Moshe, G. Alex Mason, Joel Rodriguez-Medina, Dorota Kawa, Germain Pauluzzi, et al. 2021. “Innovation, Conservation, and Repurposing of Gene Function in Root Cell Type Development.” Cell 184 (12): 3333-48.e19.
Kamiya, Takehiro, Monica Borghi, Peng Wang, John M. C. Danku, Lothar Kalmbach, Prashant S. Hosmani, Sadaf Naseer, Toru Fujiwara, Niko Geldner, and David E. Salt. 2015. “The MYB36 Transcription Factor Orchestrates Casparian Strip Formation.” Proceedings of the National Academy of Sciences of the United States of America 112 (33): 10533-38.
Lee, Yuree, Maria C. Rubio, Julien Alassimone, and Niko Geldner. 2013. “A Mechanism for Localized Lignin Deposition in the Endodermis.” Cell. https://doi.org/10.1016/j.cell.2013.02.045.
Liberman, Louisa M., Erin E. Sparks, Miguel A. Moreno-Risueno, Jalean J. Petricka, and Philip N. Benfey. 2015. “MYB36 Regulates the Transition from Proliferation to Differentiation in the Arabidopsis Root.” Proceedings of the National Academy of Sciences of the United States of America 112 (39): 12099-104.
Li, Pengxue, Meina Yang, Jiang Chang, Junqing Wu, Fenglin Zhong, Abidur Rahman, Haiyang Qin, and Shuang Wu. 2018. “Spatial Expression and Functional Analysis of Casparian Strip Regulatory Genes in Endodermis Reveals the Conserved Mechanism in Tomato.” Frontiers in Plant Science 9 (June): 832.
Liu, Jingying, Anne Osbourn, and Pengda Ma. 2015. “MYB Transcription Factors as Regulators of Phenylpropanoid Metabolism in Plants.” Molecular Plant 8 (5): 689-708.
Ma, Jian Feng, Naoki Yamaji, Namiki Mitani. Kazunori Tamai, Saeko Konishi, Toru Fujiwara, Maki Katsuhara, and Masahiro Yano. 2007. “An Efflux Transporter of Silicon in Rice.” Nature. https://doi.org/10.1038/nature05964.
Miyamoto, Takuji, Yuki Tobimatsu, and Toshiaki Umezawa. 2020. “MYB-Mediated Regulation of Lignin Biosynthesis in Grasses.” Current Plant Biology. https://doi.org/10.1016/j.cpb.2020.100174.
Nakajima, K., G. Sena, T. Nawy, and P. N. Benfey. 2001. “Intercellular Movement of the Putative Transcription Factor SHR in Root Patterning.” Nature 413 (6853): 307-11.
Nakayama, Takuya, Hidefumi Shinohara, Mina Tanaka, Koki Baba, Mari Ogawa-Ohnishi, and Yoshikatsu Matsubayashi. 2017. “A Peptide Hormone Required for Casparian Strip Diffusion Barrier Formation in Arabidopsis Roots.” Science 355 (6322): 284-86.
Naseer, S., Y. Lee, C. Lapierre, R. Franke, C. Nawrath, and N. Geldner. 2012. “Casparian Strip Diffusion Barrier in Arabidopsis Is Made of a Lignin Polymer without Suberin.” Proceedings of the National Academy of Sciences. https://doi.org/10.1073/pnas.1205726109.
Ortiz-Ramirez, Carlos, Bruno Guillotin, Xiaosa Xu, Ramin Rahni, Sanqiang Zhang, Zhe Yan. Poliana Coqueiro Dias Araujo, et al. 2021. “Ground Tissue Circuitry Regulates Organ Complexity in Maize and.” Science 374 (6572): 1247-52.
Pernas, Monica, Eoin Ryan, and Liam Dolan. 2010. “SCHIZORIZA Controls Tissue System Complexity in Plants.” Current Biology. CB 20 (9): 818-23.
Perumalla, Christopher J., Jerry G. Chmielewski, and Carol A. Peterson. 1990. “A Survey of Angiosperm Species to Detect Hypodermal Casparian Bands. Ill. Rhizomes.” Botanical Journal of the Linnean Society. https://doi.org/10.1111/j.1095-8339.1990.tb00178.x.
Perumalla, Christopher J., Carol A. Peterson, and Daryl E. Enstone. 1990. “A Survey of Angiosperm Species to Detect Hypodermal Casparian Bands. 1. Roots with a Uniseriate Hypodermis and Epidermis.” Botanical Journal of the Linnean Society. https://doi.org/10.1111/j.1095-8339.1990.tb00176.x.
Peterson, Carol A., and Christopher J. Perumalla. 1990. “A Survey of Angiosperm Species to Detect Hypodermal Casparian Bands. II. Roots with a Multiseriate Hypodermis or Epidermis.” Botanical Journal of the Linnean Society. https://doi.org/10.1111/j.1095-8339.1990.tb00177.x.
Pfister, Alexandre, Marie Barberon, Julien Alassimone, Lothar Kalmbach, Yuree Lee, Joop E. M. Vermeer, Misako Yamazaki, et al. 2014. “A Receptor-like Kinase Mutant with Absent Endodermal Diffusion Barrier Displays Selective Nutrient Homeostasis Defects.” eLife 3 (September): e03115.
Reyt, Guilhem, Priya Ramakrishna, Isai Salas-González, Satoshi Fujita, Ashley Love, David Tiemessen, Catherine Lapierre, et al. 2021. “Two Chemically Distinct Root Lignin Barriers Control Solute and Water Balance.” Nature Communications 12 (1): 2320,
Rojas-Murcia, Nelson, Kian Hématy, Yuree Lee, Aurélia Emonet, Robertas Ursache, Satoshi Fujita, Damien De Bellis, and Niko Geldner, n.d. “High-Order Mutants Reveal an Essential Requirement for Peroxidases but Not Laccases in Casparian Strip Lignification.” https://doi.org/10.1101/2020.06.17.154617.
Ron, Mily, Michael W. Dorrity, Miguel de Lucas, Ted Toal, R. Ivan Hernandez, Stefan A. Little, Julin N. Maloof, Daniel J. Kliebenstein, and Siobhan M. Brady. 2013. “Identification of Novel Loci Regulating Interspecific Variation in Root Morphology and Cellular Development in Tomato.” Plant Physiology 162 (2): 755-68.
Ron, Mily, Kaisa Kajala, Germain Pauluzzi, Dongxue Wang, Mauricio A. Reynoso, Kristina Zumstein, Jasmine Garcha, et al. 2014. “Hairy Root Transformation Using Agrobacterium Rhizogenes as a Tool for Exploring Cell Type-Specific Gene Expression and Function Using Tomato as a Model.” Plant Physiology 166 (2): 455-69.
Roppolo, Daniele, Brigitte Boeckmann, Alexandre Pfister, Emmanuel Boutet, Maria C. Rubio, Valérie Dénervaud-Tendon, Joop E. M. Vermeer, Jacqueline Gheyselinck, Ioannis Xenarios, and Niko Geldner, 2014. “Functional and Evolutionary Analysis of the CASPARIAN STRIP MEMBRANE DOMAIN PROTEIN Family.” Plant Physiology 165 (4): 1709-22.
Roppolo, Daniele, Bert De Rybel, Valérie Dénervaud Tendon, Alexandre Pfister, Julien Alassimone, Joop E. M. Vermeer, Misako Yamazaki, York-Dieter Stierhof, Tom Beeckman, and Niko Geldner. 2011. “A Novel Protein Family Mediates Casparian Strip Formation in the Endodermis.” Nature. https://doi.org/10.1038/nature10070.
Sasaki, Akimasa, Naoki Yamaji. Kengo Yokosho, and Jian Feng Ma. 2012. “Nramp5 Is a Major Transporter Responsible for Manganese and Cadmium Uptake in Rice.” The Plant Cell. https://doi.org/10.1105/tpc.112.096925.
Schalk, M., F. Cabello-Hurtado, M. A. Pierrel, R. Atanossova, P. Saindrenan, and D. Werck-Reichhart. 1998. “Piperonylic Acid, a Selective, Mechanism-Based Inactivator of the Trans-Cinnamate 4-Hydroxylase: A New Tool to Control the Flux of Metabolites in the Phenylpropanoid Pathway.” Plant Physiology 118 (1): 209-18.
Shukla, Vinay, Jian-Pu Han, Fabienne Cléard, Linnka Lefebvre-Legendre, Kay Gully, Paulina Flis, Alice Berhin, et al. 2021. “Suberin Plasticity to Developmental and Exogenous Cues Is Regulated by a Set of MYB Transcription Factors.” Proceedings of the National Academy of Sciences of the United States of America 118 (39). https://doi.org/10.1073/pnas.2101730118.
Soukup, Ales, Olga Votrubova, and Hana Cizkova. 2002. “Development of Anatomical Structure of Roots of Phragmites Australis.” New Phytologist. https://doi.org/10.1046/j.0028-646x.2001.00317.x.
Wang, Zhigang, Naoki Yamaji, Sheng Huang, Xiang Zhang, Mingxing Shi, Shan Fu, Guangzhe Yang, Jian Feng Ma, and Jixing Xia. 2019. “OsCASP1 Is Required for Casparian Strip Formation at Endodermal Cells of Rice Roots for Selective Uptake of Mineral Elements.” The Plant Cell 31 (11): 2636-48.
Meyer C J, Peterson C A. 2013. “In Plant Roots:The Hidden Half”, ed. A Eshel, T Beeckman. Boca Raton, FL: CRC. 4th edition.
Vuillemin P. 1886. L'exoderme (The exodermis). Bulletin de la Sociktk Botanigue de France, 338&84.

Illustrative Sequences

SEQ ID NO: 1 Accession number XP_019071045 zinc finger portein 10 (Solanum
lycopersicum) Solyc09g011120.1.1 SLEXO1 polypeptide sequence

| | |
|---|---|
| MEQTRYWMST KRKHDMTLSN NPSSYGDSWE EQAFAEDAAG ALGGCIWPPR SYTCSFCRRE | 60 |
| FRSAQALGGH MNVHRRDRAR MKQSPPSNSP SVHDHQVFIP PTPPLHSNHH HNSHVQYPSQ | 120 |
| ICNTFMYNPN SDSASGVPIR VSSQKTLETH HHSSLSSIVH EEKKNSLSSS WSNLVADKYS | 180 |
| CLSSVKNDEE KKMKSIDFKK DQERNLEVMI DSSFRAKHDH IVEPNYKERK VDQEDNISFA | 240 |
| NLFPRTSSSI ERCRPQSEAL ERIPSAIEEL DLELKL* | |

SEQ ID NO: 2 Solyc09g011120.1.1 cDNA sequence
ATGGAGCAAACAAGATATTGGATGAGCACAAAGAGAAAACATGACATGACATTATCAAAT
AATCCTTCATCGTATGGCGATTCGTGGGAGGAACAAGCTTTTGCTGAAGATGCAGCTGGA
GCACTTGGAGGGTGTATATGGCCACCAAGATCTTATACTTGTAGCTTTTGTAGAAGAGAA
TTTAGGTCAGCTCAAGCTCTAGGTGGCCATATGAATGTTCATAGAAGGGATAGAGCAAGA
ATGAAACAATCTCCTCCATCTAATAGTCCAAGTGTTCATGATCATCAAGTATTTATTCCT
CCTACTCCTCCTCTTCATTCTAATCATCATCACAATAGCCATGTTCAATACCCTTCTCAA
ATTTGTAATACCTTTATGTATAACCCTAATTCTGACTCTGCCTCTGGGGTTCCAATTAGG
GTTTCTTCTCAAAAGACCTTAGAAACTCATCATCATTCTTCTTTGTCATCAATTGTCCAT
GAAGAGAAAAAGAATAGCTTGTCTTCATCATGGTCAAACTTGGTGGCTGACAAATATTCT
TGTCTCTCTAGTGTGAAAAATGATGAAGAGAAGAAGATGAAATCTATAGATTTCAAAAAA
GATCAAGAGAGGAATCTTGAAGTCATGATAGACTCAAGCTTTAGGGCAAAACATGACCAT
ATTGTTGAACCTAATTACAAAAGGAGGAAAGTTGATCAAGAAGATAACATTTCATTTGCA
AATCTCTTCCCTAGAACAAGTTCATCAATCGAAAGGTGTCGTCCCCAATCAGAAGCACTT
GAAAGGATCCCTAGTGCAATAGAAGAATTGGATCTTGAGTTGAAGCTTTGA
SEQ ID NO: 3 Accession number XP004238144.1 polypeptide sequence
MALMLDNCEG ILLSLDSHKS VPAPFLTKTY QLVDDPSTDH IVSWGEDDST FVVWRPPEFA
RDLLPNYFKH NNFSSFVRQL NTYGFRKIVP DRWEFANEFF KRGEKHLLCE IHRRKTAQPV
QSMSVNHPHS YHTGSGFFPN YPSNYNPRLS VSPPDSDDQL YQQQNINWCD SPCSNNNNAS
NNNTNTVTAL SEDNDRLRRS NNMLMSELAH MRKLYNDIIY FVQNHVKPVT PSSSYNTCSL
LPASATPIVQ KNMNIHHHQF GYQQITNPKN IVISNINTNN IVSPSKTSQS SSVTILDEGN
GGDNTSTKLF GVPLMSKKRV HPEYSSSYYS TTNMVEKNKA ELMVLEKNDL GLNLMPPSSS
SEQ ID NO: 4 XM_004238096.4 cDNA sequence
ACCTCAAAAT AACCTCCTTA TTATTACCTC CCTCATTCTC ACATCTTGTT AACATTTTTT
CTTGTCATCT TCAATATTCA AGAATTTTCT TTGTTTCAAA TGGCTTTAAT GCTAGATAAT
TGTGAAGGCA TATTACTTTC ATTAGACTCA CATAAATCAG TTCCAGCTCC ATTTTTAACA
AAAACATATC AACTTGTIGA TGACCCTTCT ACTGACCATA TIGTTTCTTG GGGTGAAGAT
GATTCTACTT TTGTTGTTTG GCGTCCACCT GAATTTGCTC GTGATTTACT CCCTAATTAC
TTCAAACACA ACAATTTCTC CAGCTTCGTA CGTCAACTCA ATACTTATGG TTTCAGAAAA
ATCGTACCGG ATCGATGGGA GTTCGCTAAT GAGTTTTTCA AGAGAGGCGA AAAGCATTTA
TTATGCGAAA TCCATCGGAG AAAAACAGCT CAGCCAGTAC AAAGTATGTC AGTGAATCAT
CCCCATTCAT ATCATACGGG TTCGGGTTTT TTCCCAAATT ACCCGAGTAA TTATAACCCG
CGGCTGAGTG TTTCTCCACC TGATTCGGAT GATCAATTAT ATCAACAACA AAATATCAAT
TGGTGTGATT CGCCTTGTTC CAACAACAAT AATGCGAGCA ACAACAACAC CAATACAGTA
ACGGCGTTGT CGGAGGACAA CGACCGGTTA CGTAGAAGCA ACAATATGTT AATGTCGGAA
CTTGCACACA TGAGGAAACT TTATAACGAC ATTATTTATT TCGTTCAAAA TCATGTCAAA
CCTGTTACAC CTAGCAGTTC GTACAATACT TGTTCACTAT TACCTGCTTC AGCTACACCT
ATAGTCCAGA AGAATATGAA TATTCATCAT CATCAGTTTG GGTATCAACA AATTACAAAC
CCTAAAAATA TTGTTATATC AAACATTAAC ACCAACAACA TCGTATCACC AAGCAAGACA
TCACAAAGCA GTAGCGTGAC GATACTTGAT GAAGGTAATG GTGGTGATAA TACAAGTACA
AAATTATTTG GTGTTCCTCT TATGTCCAAG AAAAGAGTGC ATCCTGAATA TTCATCTTCA
TATTATTCGA CTACGAATAT GGTGGAGAAA AATAAGGCAA GATTAATGGT ATTGGAAAAA
AATGATCTAG GGTTAAATCT CATGCCTCCT TCTTCATCTT AGACTTTATT TTTTTACTAT
TATTATTCAC CCACACTACC TTATTAAAGT TCAAGTTTTT GTTTCATTAG TGGATCA

What is claimed is:

1. A method of generating a plant having enhanced lignification of root exodermis, the method comprising (a) genetically modifying a gene encoding a SlEXO zinc finger transcription factor to reduce or knockout expression of the SlEXO transcription factor; and (b) genetically modifying a gene encoding a heat shock transcription factor B4 Scz transcription factor to reduce or knockout expression of the Scz transcription factor; wherein the plant has enhanced lignification of root exodermis compared to a counterpart control plant.

2. The method of claim 1, wherein (a) comprises gene editing of the gene encoding the SLEXO zinc finger transcription factor and/or (b) comprises gene editing of the gene encoding the Scz

3. The method of claim 2, wherein gene editing comprises CRISPR Cas gene editing.

4. The method of claim 1, wherein (a) comprises introducing an inhibitory RNA that targets a polynucleotide encoding the SLEXO zinc finger transcription factor and/or (b) comprises introducing an inhibitory RNA that targets a polynucleotide encoding the the Scz transcription factor.

5. The method of any one of the preceding claims, wherein the polypeptide encoded by the gene encoding SlEXO has at least 75% identity to SEQ ID NO:1 and/or the polypeptide encoded by the Scz gene has at least 75% identity to SEQ ID NO:3.s

6. The method of any one of the preceding claims, wherein the plant is a solanaceous plant.

7. The method of claim 6, wherein the plant is a tomato plant.

8. A plant generated by the method of any one of claims 1-7.

9. A plant having increased having increased drought and/or salt tolerance, compared to a counterpart control plant, wherein the plant (a) is mutated or engineered to reduce or knockout expression of a gene encoding a SlEXO zinc finger transcription factor and (b) mutated or engineered to reduce or knockout expression of a gene encoding a heat shock transcription factor B4 Scz transcription factor.

10. The plant of claim 9, wherein the plant of (a) is mutated or engineered to reduce or knockout expression of, or expresses an siRNA or antisense polynucleotide to reduce expression of, the SLEXO transcription factor, or expresses a modified endonuclease such as Cas9 targeted to reduce expression of the SLEXO transcription factor and/or the Scz transcription factor.

11. The plant of claim 9 or 10 wherein the plant is a solanaceous plant.

12. The plant of claim 11, wherein the plant is a tomato plant.


SEQUENCE LISTING (4 sequences)

SEQ ID NO: 1 - protein, 276 aa, Solanum lycopersicum
MEQTRYWMSTKRKHDMTLSNNPSSYGDSWEEQAFAEDAAGALGGCIWPPRSYTCSFCRREFRSAQALGGHMNVHRRDRARMKQSPPSNSPSVHDHQVFIPPTPPLHSNHHHNSHVQYPSQICNTFMYNPNSDSASGVPIRVSSQKTLETHHHSSLSSIVHEEKKNSLSSSWSNLVADKYSCLSSVKNDEEKKMKSIDFKKDQERNLEVMIDSSFRAKHDHIVEPNYKRRKVDQEDNISFANLFPRTSSSIERCRPQSEALERIPSAIEELDLELKL

SEQ ID NO: 2 - DNA, 831 bp, synthetic construct
  source 1..831: mol_type = other DNA
atggagcaaacaagatattggatgagcacaaagagaaaacatgacatgacattatcaaataatccttcatcgtatggcgattcgtgggaggaacaagcttttgctgaagatgcagctggagcacttggagggtgtatatggccaccaagatcttatacttgtagcttttgtagaagagaatttaggtcagctcaagctctaggtggccatatgaatgttcatagaagggatagagcaagaatgaaacaatctcctccatctaatagtccaagtgttcatgatcatcaagtatttattcctcctactcctcctcttcattctaatcatcatcacaatagccatgttcaatacccttctcaaatttgtaatacctttatgtataaccctaattctgactctgcctctggggttccaattagggtttcttctcaaaagaccttagaaactcatcatcattcttctttgtcatcaattgtccatgaagagaaaaagaatagcttgtcttcatcatggtcaaacttggtggctgacaaatattcttgtctctctagtgtgaaaaatgatgaagagaagaagatgaaatctatagatttcaaaaaagatcaagagaggaatcttgaagtcatgatagactcaagctttagggcaaaacatgaccatattgttgaacctaattacaaaaggaggaaagttgatcaagaagataacatttcatttgcaaatctcttccctagaacaagttcatcaatcgaaaggtgtcgtccccaatcagaagcacttgaaaggatccctagtgcaatagaagaattggatcttgagttgaagctttga

SEQ ID NO: 3 - protein, 360 aa, Solanum lycopersicum
MALMLDNCEGILLSLDSHKSVPAPFLTKTYQLVDDPSTDHIVSWGEDDSTFVVWRPPEFARDLLPNYFKHNNFSSFVRQLNTYGFRKIVPDRWEFANEFFKRGEKHLLCEIHRRKTAQPVQSMSVNHPHSYHTGSGFFPNYPSNYNPRLSVSPPDSDDQLYQQQNINWCDSPCSNNNNASNNNTNTVTALSEDNDRLRRSNNMLMSELAHMRKLYNDIIYFVQNHVKPVTPSSSYNTCSLLPASATPIVQKNMNIHHHQFGYQQITNPKNIVISNINTNNIVSPSKTSQSSSVTILDEGNGGDNTSTKLFGVPLMSKKRVHPEYSSSYYSTTNMVEKNKARLMVLEKNDLGLNLMPPSSS

SEQ ID NO: 4 - DNA, 1257 bp, synthetic construct
  source 1..1257: mol_type = other DNA
acctcaaaataacctccttattattacctccctcattctcacatcttgttaacattttttcttgtcatcttcaatattcaagaattttctttgtttcaaatggctttaatgctagataattgtgaaggcatattactttcattagactcacataaatcagttccagctccatttttaacaaaaacatatcaacttgttgatgacccttctactgaccatattgtttcttggggtgaagatgattctacttttgttgtttggcgtccacctgaatttgctcgtgatttactccctaattacttcaaacacaacaatttctccagcttcgtacgtcaactcaatacttatggtttcagaaaaatcgtaccggatcgatgggagttcgctaatgagtttttcaagagaggcgaaaagcatttattatgcgaaatccatcggagaaaaacagctcagccagtacaaagtatgtcagtgaatcatccccattcatatcatacgggttcgggttttttcccaaattacccgagtaattataacccgcggctgagtgtttctccacctgattcggatgatcaattatatcaacaacaaaatatcaattggtgtgattcgccttgttccaacaacaataatgcgagcaacaacaacaccaatacagtaacggcgttgtcggaggacaacgaccggttacgtagaagcaacaatatgttaatgtcggaacttgcacacatgaggaaactttataacgacattatttatttcgttcaaaatcatgtcaaacctgttacacctagcagttcgtacaatacttgttcactattacctgcttcagctacacctatagtccagaagaatatgaatattcatcatcatcagtttgggtatcaacaaattacaaaccctaaaaatattgttatatcaaacattaacaccaacaacatcgtatcaccaagcaagacatcacaaagcagtagcgtgacgatacttgatgaaggtaatggtggtgataatacaagtacaaaattatttggtgttcctcttatgtccaagaaaagagtgcatcctgaatattcatcttcatattattcgactacgaatatggtggagaaaaataaggcaagattaatggtattggaaaaaaatgatctagggttaaatctcatgcctccttcttcatcttagactttatttttttactattattattcacccacactaccttattaaagttcaagtttttgtttcattagtggatca